(19) United States
(12) Patent Application Publication
(10) Pub. No.: US 2026/0187959 A1
(43) Pub. Date: Jul. 2, 2026

(54) ENDOSCOPE APPARATUS, IMAGE PROCESSING DEVICE, IN-HOSPITAL SYSTEM, IMAGE PROCESSING METHOD, AND COMPUTER-READABLE STORAGE MEDIUM

(71) Applicant: OLYMPUS MEDICAL SYSTEMS CORP., Tokyo (JP)

(72) Inventors: Koichi SHINTANI, Tokyo (JP); Kazuo KANDA, Tokyo (JP); Manabu ICHIKAWA, Tokyo (JP); Satoshi YOSHIDA, Tokyo (JP); Shunsuke ROKUTANI, Tokyo (JP); Hiroshi KUBO, Tokyo (JP); Osamu NONAKA, Tokyo (JP)

(73) Assignee: OLYMPUS MEDICAL SYSTEMS CORP., Tokyo (JP)

(21) Appl. No.: 19/549,781

(22) Filed: Feb. 25, 2026

Related U.S. Application Data
(63) Continuation of application No. PCT/JP2023/032028, filed on Aug. 31, 2023.

(51) Int. Cl. G06V 10/24; A61B 1/00; A61B 1/05; A61B 1/273; G06V 10/10; G06V 10/25; G06V 10/32; G16H 40/67

(52) U.S. Cl. CPC G06V 10/242; A61B 1/000095; G06V 10/16; G06V 10/25; G06V 10/32; G16H 40/67; A61B 1/05; A61B 1/2733; G06V 2201/03

(57) ABSTRACT

An endoscope apparatus includes an endoscope and processing circuitry. The endoscope includes an imaging device disposed at a distal end of an insertion portion and configured to capture endoscopic images when the endoscope is inserted into a human body. The processing circuitry is configured to determine, based on image changes of the endoscopic images, whether each endoscopic image is a non-observation target image or an observation target image, detect a specific region based on image characteristics in endoscopic images determined as the non-observation target images, determine images including the specific region among the non-observation target images as correction target images, determine a directional relationship between a direction of the imaging device and an anatomical orientation of the human body, based on the correction target images, and

perform direction standardization processing based on the directional relationship, thereby standardizing orientations of the correction target images based on the anatomical orientation.

### CROSS REFERENCE TO RELATED APPLICATION

This application is a continuation of International Application No. PCT/JP2023/032028, filed on Aug. 31, 2023, the entire contents of which are incorporated herein by reference.

### FIELD

The present disclosure relates to an endoscope apparatus, an image processing device, an in-hospital system, an image processing method, and a non-transitory computer-readable storage medium, which are adapted to use an image obtained during insertion of an endoscope for observation and treatment.

### BACKGROUND

An endoscope is a device that is inserted into a body or the like to enable observation of a diseased part or the like that cannot be seen from the outside. The endoscope includes an elongated and flexible insertion portion that is inserted into a body cavity of a patient, for example. The endoscope picks up an image of an observation target region by an image pickup device provided at a distal end of the insertion portion. The image (endoscopic image) obtained by being picked up with the endoscope is supplied to a video processor. The endoscopic image processed by the video processor is displayed on a display screen of a monitor.

It is relatively difficult to insert such an endoscope into a lumen of a human body. Therefore, observation and treatment using the endoscope may be, for example, in a case of an examination of the large intestine or the like, sometimes performed while the insertion portion is withdrawn after the insertion portion has first reached the observation target region. When an endoscope is inserted into the upper digestive tract, the lower digestive tract or the like, it is necessary to insert the endoscope in accordance with the shape of the digestive tract, which may be narrow in some areas.

At this time, a physician advances or retracts, and twists the insertion portion, to insert the insertion portion into the observation target region. Therefore, in particular during insertion of the endoscope, the physician does not have enough room for observation, and the images acquired are also often unsuitable for observation.

In this manner, since endoscopic images are picked up by changing an orientation of a distal end portion located away from the physician's hand, Japanese Patent Application Laid-Open Publication No. 2010-220794 (hereinafter, referred to as Patent Literature 1) discloses a technology for automatically aligning an orientation of an object region reflected in a plurality of endoscopic images of the same object region picked up at different time points.

### SUMMARY

According to aspects of the present disclosure, an endoscope apparatus is provided, which includes an endoscope and processing circuitry. The endoscope includes an imaging device disposed at a distal end of an insertion portion and configured to capture a plurality of endoscopic images when the endoscope is inserted into a human body. The processing circuitry is configured to, based on image changes of the plurality of endoscopic images, determine whether each endoscopic image is a non-observation target image or an observation target image. The processing circuitry is further configured to detect a specific region based on image characteristics in endoscopic images determined as the non-observation target images. The processing circuitry is further configured to determine images including the specific region among the non-observation target images as correction target images. The processing circuitry is further configured to determine a directional relationship between a direction of the imaging device and an anatomical orientation of the human body, based on the correction target images. The processing circuitry is further configured to perform direction standardization processing to successively rotate the correction target images obtained sequentially during the insertion of the endoscope based on the directional relationship, thereby standardizing orientations of the correction target images based on the anatomical orientation as a reference.

According to aspects of the present disclosure, further provided is an image processing device that includes a connection interface and processing circuitry. The processing circuitry is configured to receive, via the connection interface, a plurality of endoscopic images captured by an imaging device of an endoscope when the endoscope is inserted into a human body. The processing circuitry is further configured to, based on image changes of the plurality of endoscopic images, determine whether each endoscopic image is a non-observation target image or an observation target image. The processing circuitry is further configured to detect a specific region based on image characteristics in endoscopic images determined as the non-observation target images. The processing circuitry is further configured to determine images including the specific region among the non-observation target images as correction target images. The processing circuitry is further configured to determine a directional relationship between a direction of the imaging device and an anatomical orientation of the human body, based on each correction target image. The processing circuitry is further configured to perform direction standardization processing to successively rotate the correction target images obtained sequentially during the insertion of the endoscope based on the directional relationship, thereby standardizing orientations of the correction target images based on the anatomical orientation as a reference.

According to aspects of the present disclosure, further provided is an in-hospital system that includes a receiving device and processing circuitry. The receiving device is configured to receive, from an endoscope, temporally continuous endoscopic images captured by an imaging device. The endoscopic images include non-observation target images and observation target images. The processing circuitry is configured to detect a specific region based on image characteristics in the non-observation target images. The processing circuitry is further configured to determine images including the specific region among the non-observation target images as correction target images. The processing circuitry is further configured to perform rotational correction on each of the temporally continuous correction target images based on the specific region in the correction target images. The processing circuitry is further configured to associate the images subjected to the rotational correction with text related to a symptom of the specific region as an examination result.

According to aspects of the present disclosure, further provided is an image processing method implementable by processing circuitry configured to be connected to an endoscope. The image processing method includes receiving, from the endoscope, temporally continuous endoscopic images captured by an imaging device. The endoscopic images include non-observation target images and observation target images. The image processing method further includes detecting a specific region based on image characteristics in the non-observation target images. The image processing method further includes determining images including the specific region among the non-observation target images as correction target images. The image processing method further includes performing rotational correction on each of the temporally continuous correction target images based on the specific region in each correction target image. The image processing method further includes associating the images subjected to the rotational correction with text related to a symptom of the specific region as an examination result.

According to aspects of the present disclosure, further provided is an image processing method implementable by processing circuitry configured to be connected to an endoscope. The image processing method includes receiving a plurality of endoscopic images captured by an imaging device of the endoscope when the endoscope is inserted into a human body. The image processing method further includes determining, based on image changes of the plurality of endoscopic images, whether each endoscopic image is a non-observation target image or an observation target image. The image processing method further includes detecting a specific region based on image characteristics in endoscopic images determined as the non-observation target images. The image processing method further includes determining images including the specific region among the non-observation target images as correction target images. The image processing method further includes determining a directional relationship between a direction of the imaging device and an anatomical orientation of the human body, based on each correction target image. The image processing method further includes performing direction standardization processing to successively rotate the correction target images sequentially obtained during the insertion of the endoscope based on the directional relationship, thereby standardizing orientations of the correction target images based on the anatomical orientation as a reference.

A non-transitory computer-readable storage medium storing computer-readable instructions that are executable by processing circuitry configured to be connected to an endoscope and are configured to, when executed by the processing circuitry, cause the processing circuitry to perform an image processing method. The image processing method includes receiving a plurality of endoscopic images captured by an imaging device of the endoscope when the endoscope is inserted into a human body. The image processing method further includes determining, based on image changes of the plurality of endoscopic images, whether each endoscopic image is a non-observation target image or an observation target image. The image processing method further includes detecting a specific region based on image characteristics in endoscopic images determined as the non-observation target images. The image processing method further includes determining images including the specific region among the non-observation target images as correction target images. The image processing method further includes determining a directional relationship between a direction of the imaging device and an anatomical orientation of the human body, based on each correction target image. The image processing method further includes performing direction standardization processing to successively rotate the correction target images sequentially obtained during the insertion of the endoscope based on the directional relationship, thereby standardizing orientations of the correction target images based on the anatomical orientation as a reference.

### BRIEF DESCRIPTION OF DRAWINGS

FIG. 1 is a configuration diagram showing an endoscope apparatus according to a first embodiment of the present disclosure.

FIG. 2 is an explanatory diagram showing the oral cavity, the nasal cavity, the larynx, and the pharynx portion of a human body.

FIG. 3 is an explanatory diagram for explaining an anatomical orientation.

FIG. 4 is a flowchart for explaining an operation of the first embodiment.

FIG. 5 is an explanatory diagram for explaining insertion of an endoscope.

FIG. 6 is an explanatory diagram showing an example of a relationship between an endoscopic image and the anatomical orientation.

FIG. 7 is an explanatory diagram for explaining a method for determining a relationship between a specific region and the anatomical orientation.

FIG. 8 is an explanatory diagram showing an endoscopic image that is acquired.

FIG. 9 is an explanatory diagram showing a display example.

FIG. 10 is an explanatory diagram showing an example of the specific regions.

FIG. 11 is an explanatory diagram showing an example of the specific regions.

FIG. 12 is an explanatory diagram showing an example of the specific regions.

FIG. 13 is a flowchart showing an operation flow adopted in a second embodiment.

FIG. 14 is an explanatory diagram for explaining an image synthesis in the second embodiment.

FIG. 15 is an explanatory diagram for explaining the image synthesis in the second embodiment.

FIG. 16 is a flowchart showing a modification example of the second embodiment.

FIG. 17 is a block diagram showing a third embodiment.

FIG. 18 is a flowchart for explaining an operation of the third embodiment.

FIG. 19 is a flowchart for explaining the operation of the third embodiment.

FIG. 20 is a flowchart for explaining the operation of the third embodiment.

### DESCRIPTION OF EMBODIMENTS

During insertion of an endoscope, or the like, images obtained when the physician is concentrating on an insertion operation into a narrow and winding lumen are not intended for observation (endoscope distal end portion moving process images or position change progress images) and are therefore unsuitable for observation. Furthermore, even if the technology disclosed in Patent Literature 1 is applied, it is difficult to use the images obtained during insertion of the endoscope for observation or treatment.

The specification describes, for example, an endoscope apparatus, an image processing device, an in-hospital system, an image processing method, and an image processing program that enable images obtained when a physician is concentrating on an insertion operation during insertion of an endoscope, or the like (in other words, progress images that were not originally intended to be used for observation or treatment), to be easily used for observation or treatment of each region. Of course, it is possible to make it easier to use images obtained during preparation, not for observation, such as in an operation situation when removing the endoscope from a narrow lumen other than inserting into the lumen, or when changing a position of a distal end of the endoscope, for observation or treatment of each region.

According to the disclosure of the specification, for example, it is possible to make it easier to use position change progress images (endoscope distal end portion moving process images) obtained by changing a distal end position of an endoscope during a process such as an observation preparation or a treatment preparation, for observation and treatment of each region.

Hereinafter, embodiments of the present disclosure will be described in detail with reference to the drawings.

### First Embodiment

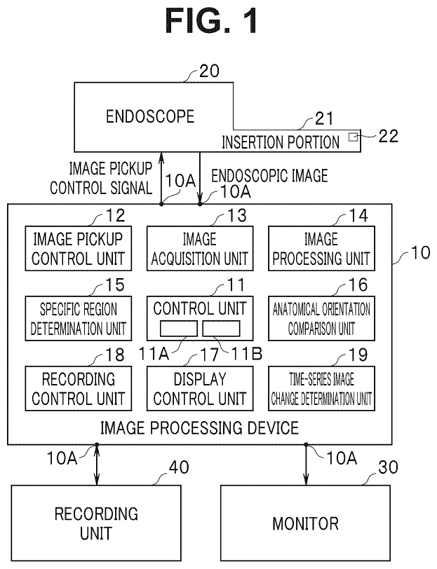

FIG. 1 is a configuration diagram showing an endoscope apparatus according to a first embodiment of the present disclosure. In the present embodiment, from endoscopic images such as endoscope distal end portion moving process images or position change progress images, which are acquired by an endoscope during a process such as an observation preparation or a treatment preparation by changing a position of a distal end of the endoscope when the endoscope (insertion portion) is inserted, a relationship between an orientation of a display of a specific region of a human body included in the endoscopic image on a screen and an anatomical orientation of the specific region is obtained, to enable vertical and horizontal directions of the endoscopic images to be displayed on the screen to be aligned based on the anatomical orientation. Furthermore, the present embodiment makes it easier to use the endoscopic images acquired in the position change progress of the endoscope for observation, treatment, or the like by performing processing to improve an image quality of the specific region in the endoscopic images. Note that the endoscopic image includes, in addition to the endoscope distal end portion moving process image and the position change progress image, an observation image and an examination image for observing and examining a diseased part or the like, an image during treatment when performing some kind of treatment, and the like. The difference among these images appears as differences in images of a same target taken in series of frames (observation image, examination image, image during treatment), images of different targets taken in a time-series of frames, or images that change in a direction of a hole of a lumen (endoscope distal end portion moving process image, position change progress image). That is, it can be determined by checking characteristics of continuous images in time-series (target pattern, change in shadows).

In FIG. 1, endoscopic images from an endoscope 20 are inputted to an image processing device 10. The endoscope 20 includes an image pickup device 22. The image pickup device 22 is, for example, provided at a distal end of an insertion portion 21. The endoscope 20 includes an optical system (not shown) that guides an object optical image to an image pickup surface of the image pickup device 22. The optical system and the image pickup device 22 constitute an imaging device. The image pickup device 22 includes a CCD sensor, a CMOS sensor, or the like, and photoelectrically converts the object optical image from the optical system to acquire a picked-up image (image pickup signal) of an object. Note that the optical system may include lenses, apertures, or the like (not shown) for zooming and focusing, and may include a zoom (magnification) mechanism, a focus and aperture mechanism, which are not shown, for driving the lenses. Image information of the picked-up images (endoscopic images) obtained by image pickup with the image pickup device 22 is supplied to the image processing device 10.

The image processing device 10 includes a control unit 11, an image pickup control unit 12, an image acquisition unit 13, an image processing unit 14, a specific region determination unit 15, an anatomical orientation comparison unit 16, a display control unit 17, and a recording control unit 18. The image processing device 10 further includes connection interfaces 10A and is configured to be communicably connected to external devices (e.g., the endoscope 20) via the connection interfaces 10A. The control unit 11 and each component of the image processing device 10 may be implemented by processing circuitry including one or more processors, such as a CPU (central processing unit), an FPGA (field programmable gate array), or the like, may be configured to operate in accordance with one or more programs stored in a memory and control each unit, or may realize at least one of functions with one or more hardware electronic circuits. For instance, the control unit 11 may include processing circuitry including a CPU 11A and a memory 11B, and may be configured to take overall control of the image processing device 10 by the CPU 11A executing one or more programs stored in the memory 11B.

The control unit 11 comprehensively controls the entire image processing device 10. The image pickup control unit 12 generates an image pickup control signal for controlling image pickup by the endoscope 20 and provides the image pickup control signal to the endoscope 20. The image pickup control unit 12 controls driving of the image pickup device 22, and controls zooming and focusing of the optical system (not shown). In other words, the image pickup control unit 12 can perform autofocus control of the image pickup device 22.

The image acquisition unit 13 acquires the endoscopic image, which is image information from the endoscope 20 during an endoscopy. The image processing unit 14 performs predetermined image signal processing on the endoscopic image acquired by the image acquisition unit 13. For example, the image processing unit 14 performs predetermined signal processing such as color adjustment processing, matrix conversion processing, noise removal processing, and other various signal processing, for example, on the endoscopic image acquired by the image pickup device 22. The display control unit 17 provides an image (endoscopic image) obtained by the image processing by the image processing unit 14 to a monitor 30 to cause the image to be displayed. The monitor 30 is a display device including a display screen such as an LCD (liquid crystal display device).

As described above, in the examination or the treatment using the endoscope 20, a physician operates the endoscope 20 and inserts the insertion portion 21 of the endoscope 20 into a human body. A bending portion (not shown) is provided at a distal end of the insertion portion 21, and the physician inserts the distal end of the insertion portion 21 to an observation target region by operating a bending knob (not shown) or the like provided on the endoscope 20 to bend the bending portion, or by advancing or retracting the insertion portion 21. In the endoscopy, it is relatively difficult to insert the insertion portion 21 in accordance with a state of the lumen, which is narrow or curved. Furthermore, since the left, right, top and bottom of the image obtained in the position change progress process of the distal end portion at that time are changed by a twisting operation or the like, it is difficult to observe a passage region at the same position in the image while focusing and performing other image quality improvement controls.

For this reason, images obtained during insertion of the endoscope (or during moving of the endoscope distal end) until reaching a specific examination target region are not used for diagnosis, or the like, in some cases. However, in a process of reaching such an observation target region during insertion of the endoscope, images of each region other than the observation target region are also obtained. For example, in a stomach endoscopy, images of the larynx and pharynx are also obtained during insertion. In other words, if endoscopic images obtained during insertion can be used for observation or treatment, for example, it is possible to obtain valuable information that is useful for a next examination or the like.

However, when the cylindrical endoscope distal end portion (insertion portion) is moved along the lumen or the like, the top and bottom of the image are not maintained, and the image tends to rotate clockwise or counterclockwise. Therefore, in the present application, efforts are made to align the top and bottom of the images of each region based on the anatomical characteristics of the observation region, enabling to obtain images comparable to those published in papers even during moving of the endoscope. Such images with the adjusted top and bottom facilitate detection of characteristic findings or symptoms, or the like. In addition, by focusing, exposure control, and image processing to obtain such images, it is possible to photograph clear images. By photographing, processing, and recording images similar to reference images in papers or the like, the images are normalized, so to speak, enabling comparison of the sizes of tumors, lesions, and follicles, and allowing appropriate determination, examination, and diagnosis.

Therefore, in the present embodiment, the specific region determination unit 15 and the anatomical orientation comparison unit 16 are provided. The specific region determination unit 15 determines a specific region of the human body from the endoscopic image acquired by the endoscope 20. The specific region is a region where specific anatomical characteristics can be identified, and includes not only a case where the anatomical characteristics of the specific region can be directly identified from an image of the specific region, but also a case where the anatomic characteristics of the specific region can be inferred from information of another specific region whose anatomical characteristics have been identified.

As described below for a method for determining a specific region, the specific region may be determined by referring to characteristic colors or shapes of the region, patterns such as blood vessels visible on a surface, and the like in a database provided in a recording unit. In addition, there is also a method for determining the position of the endoscope distal end portion within the body by receiving a transmission result of a magnetic transmitter or the like provided at the distal end of the endoscope by an external receiver. Representative images (images with adjusted image quality as published in the papers, or the like) of such specific regions, and data indicating the image characteristics of each region are recorded in a recording unit 40, and the specific region determination unit 15 may determine the specific region while referring to the information recorded in the recording unit 40. In addition, an image representation (such as focus, exposure, color tone, field of view, and top and bottom on the screen) when each region is photographed may be determined by referring to the information recorded in the recording unit 40. Basically, the determination, photographing, and recording of images may be controlled to ensure that the resulting images are similar to such representative images.

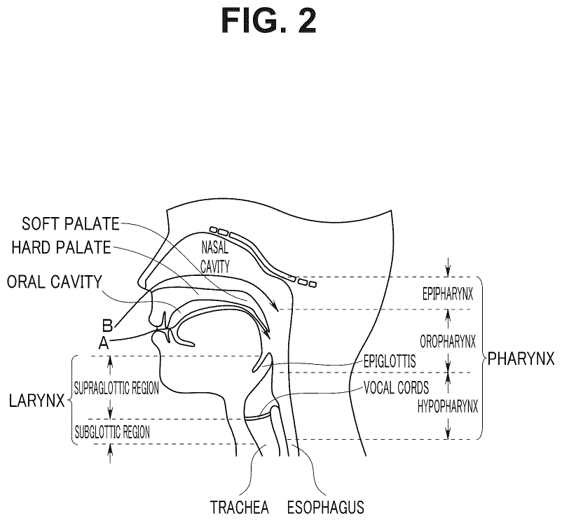

FIG. 2 is an explanatory diagram showing the oral cavity, the nasal cavity, the larynx, and the pharynx portion of the human body.

An upper gastrointestinal endoscopy includes an oral endoscopy in which the endoscope is inserted through the mouth as shown by the arrow A, and a transnasal endoscopy in which the endoscope is inserted through the nose as shown by the arrow B. In these examinations, the insertion portion 21 of the endoscope is inserted through the mouth or the nose, and advanced to reach the upper gastrointestinal tract (esophagus, stomach, and duodenum) for examination. In other words, in both examinations, the insertion portion 21 passes through various regions before reaching the esophagus. The unit of the region may be organs with specific functions, or may be a part such as an entrance or a side surface of an organ. Organs with large volumes, such as the large intestine and the stomach, have several regions to be examined, but here, parts into which the organ is divided are referred to as regions.

For example, in the oral endoscopy, when the insertion portion 21 passes through the oral cavity, the insertion portion 21 passes through the lips, teeth, and the hard palate, which blocks the passage to the nostrils when eating or drinking to inhibit food from entering the nose, then passes through the soft palate, which is a soft part at the back of the hard palate, and then passes through the epiglottis, which is located at the entrance to the trachea, in the process of reaching the esophagus from the oropharynx. The epiglottis has a function of covering the trachea during swallowing to inhibit food from entering the trachea and guiding food into the esophagus.

Furthermore, the insertion portion 21 passes through the oropharynx and the hypopharynx to reach the esophagus. Note that the esophagus and the trachea branch off in the oropharynx, and the larynx is a part that connects the oropharynx and the trachea. The larynx and the hypopharynx are adjacent to each other. The larynx is an organ known as the so-called “Adam's apple”, and separates the trachea from the pharynx, allowing air taken in through the nose and the mouth to be directed to the trachea and food and drink to be directed to the esophagus.

The larynx is not only an air passage, but also an important organ having a function that vibrates the vocal cords to produce sound. Since the larynx is adjacent to the esophagus, when the endoscope is inserted into the esophagus, images of a part of the larynx can be picked up. Even with an upper endoscope which is inserted through the nose, since the insertion portion 21 passes through the nasal cavity, the epipharynx, the oropharynx, and the hypopharynx, the upper endoscope can also pick up images of these regions during insertion and withdrawal of the upper endoscope.

An examination has been performed to check for the presence or absence of swelling and the follicles by such an observation of the pharynx, but it is also possible to diagnose infections and the like using photographed images of these regions. Furthermore, in a case of a patient affected by chronic obstructive pulmonary disease (COPD) or the like, the esophagus may be compressed due to changes in intrathoracic pressure even if only one side of the lung is abnormal, leading to a decrease in the circularity of the esophagus. The shape of the esophagus may be determined from tubular images photographed before and after the endoscope is inserted into the esophagus. In addition, as shown in FIG. 11, the respiratory organs such as the trachea and the lungs are located near a route through which the upper endoscope passes. If there are abnormalities in the respiratory organs such as the lungs, the respiratory organs and regions before and after the respiratory organs through which the endoscope passes may be affected by compression due to the intrathoracic pressure or the like, which may cause shape deformation somewhere in the lumen of the esophagus, the entrance to the bronchi, or the like, for example. In other words, information on other organs can be obtained during a process for examination of the digestive tract. Further, in addition to detecting lesions such as tumors or polyps, it is also possible to determine abnormalities of the respiratory organ based on parameters such as abnormal changes in color and abnormal changes in the shape of the esophagus. If any abnormalities are determined, it is possible to recommend bronchial examinations in the middle of the examination or as an additional examination. The determination of the distortion can be made by comparing the detected shape of the lumen with images (e.g., healthy and unhealthy) stored in a database provided in the recording unit 40, and determining the distortion based on a degree of the similarity to the unhealthy images, or by calculating the circularity of the contour of the lumen and determining the distortion numerically. The shape is compared with normal conditions (images and numerical values) inferred from general anatomical data or previous examination data, and an abnormal condition is determined when a difference in shape exceeds a threshold value set in advance.

The specific region determination unit 15 determines the specific region by image analysis and inference processing of the endoscopic images. For example, the specific region determination unit 15 can identify specific regions such as the nasal cavity and the stomach by determining the shape of the lumen from the image characteristics. In addition, for example, by determining that the insertion portion 21 advances while bending, from changes in the image characteristics (history of images), the specific region determination unit 15 can determine specific regions such as the large intestine and can also determine whether the region is the descending colon or the transverse colon, or the like.

When the specific region determination unit 15 determines that an endoscopic image is an image having characteristics specific to the specific region (an image of the specific region classified by the anatomical characteristics), the specific region determination unit 15 outputs a result of the determination to the image pickup control unit 12, the image processing unit 14, and the anatomical orientation comparison unit 16. The image pickup control unit 12 outputs the image pickup control signal for improving the image quality of the image of the specific region to the endoscope 20. For example, the image pickup control unit 12 performs tracking autofocus (AF) that performs autofocus while tracking the specific region. In addition, the image pickup control unit 12 may also perform exposure adjustment, illumination light control, or the like. The image processing unit 14 performs image processing for visibility enhancement by improving the image quality of the image of the specific region.

In addition, even for endoscopic images of the same region, the orientation of the image (the up, down, left, and right orientation of the image displayed on the display screen) varies depending on the orientation of the lumen, the position at the time of the insertion, and the like. Therefore, an anatomical position is adopted to clarify a positional relationship between each part in the human body and the endoscopic image. In addition, medical image data may also be organized using the anatomical characteristics as a reference. In other words, the medical image data subjected to direction standardization processing considering the anatomical orientation are constructed, and the medical image data of each region in the body organized based on the anatomical characteristics are constructed. This improves the visibility of the medical image data, and makes it easier to classify, organize, and compare. Note that the regions in the body organized using the anatomical characteristics as a reference are each organ of the body and constituent parts of the organ, and are assumed to be parts named in “anatomical terminology” compiled by academic societies, for example. It is assumed that the names of such regions in the body can be determined from the anatomical positions, images obtained by photographing the regions, or the like, using an image table for referring to region names and an inference model. Information on what examinations are performed may also be referred when determining the region.

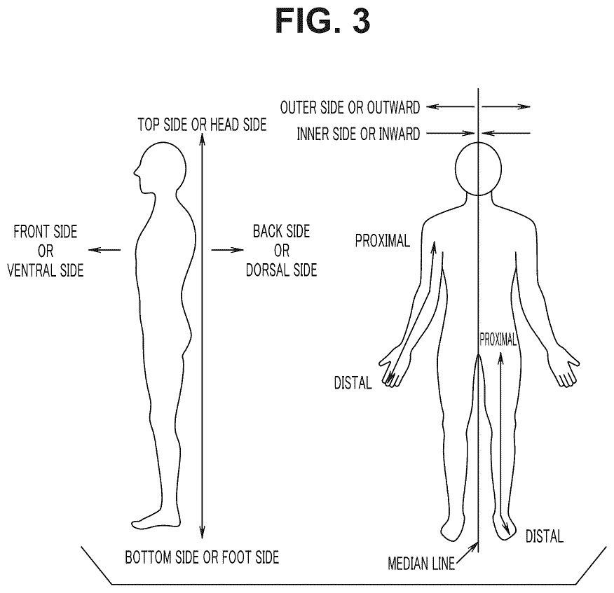

FIG. 3 is an explanatory diagram for explaining the anatomical orientation.

The anatomical position (anatomical orientation) refers to a posture in which both feet are facing forward, both arms are rotated outward, palms are facing forward, and thumbs are pointing outward. In the anatomical orientation, the front is the ventral side and the back is the dorsal side. In addition, the top is the head side and the bottom is the foot side. The center side of the body is the inner side or the inward, and the direction away from the center is the outer side or the outward. Furthermore, the base side of the arms and legs is proximal, and the fingertip sides are distal.

Here, let two directions perpendicular to each other on a plane perpendicular to an advancing direction of the insertion portion 21 be the vertical direction and the horizontal direction, respectively, for example. The image pickup device 22 is fixed at the distal end of the insertion portion 21, and the vertical and horizontal directions of the image pickup surface of the image pickup device 22 (hereinafter, referred to as the vertical and horizontal directions of the imaging device) are aligned with the vertical and horizontal directions of the insertion portion 21. When the endoscopic image acquired by the endoscope 20 is displayed on the display screen of the monitor 30 without image correction, the image whose vertical and horizontal directions are aligned with the vertical and horizontal directions of the insertion portion 21 is displayed as-is in the vertical and horizontal directions of the display screen of the monitor 30. Note that a vertical scanning direction of the monitor 30 is the vertical direction of the display screen, and a horizontal scanning direction of the monitor 30 is the horizontal direction of the display screen.

To simplify the description, the orientation of the image displayed on the display screen is described below simply as the orientation of the image. In other words, the orientation of the endoscopic image indicates a direction corresponding to the vertical and horizontal directions of the imaging device.

However, the vertical and horizontal directions of the insertion portion 21 are unrelated to the direction based on the anatomical orientation, and furthermore, when the insertion portion 21 is inserted, the insertion portion 21 is twisted, resulting in a change in the relationship between the vertical and horizontal directions of the insertion portion 21 and the direction based on the anatomical orientation. As a result, the orientation of the endoscopic image displayed on the display screen of the monitor 30 is unrelated to the anatomical orientation, and the relationship between the orientation of the endoscopic image and the anatomical orientation changes over time.

In the present embodiment, the anatomical orientation comparison unit 16 obtains the orientation of the image for the specific region in the vertical and horizontal directions based on the anatomical orientation. In other words, the anatomical orientation comparison unit 16 obtains a directional relationship between the orientation of the endoscopic image (the vertical and horizontal directions of the imaging device) and the anatomical orientation. The anatomical orientation comparison unit 16 detects the positional relationship between the specific region in the image and the anatomical orientation. It can be said that the detection result of the anatomical orientation comparison unit 16 is twist information of the insertion portion 21 based on the anatomical orientation.

The anatomical orientation comparison unit 16 determines the anatomical orientation of an image part of the specific region by image analysis of the endoscopic image. For example, the anatomical orientation comparison unit 16 can determine the relationship between each image part of the specific region and the anatomical orientation from the shape of the specific region such as the nasal cavity or the stomach. In addition, for example, the anatomical orientation comparison unit 16 can determine the relationship between each image part of the specific region and the anatomical orientation from the shape of each portion in the endoscopic image for the esophagus, the trachea and the like. Furthermore, the anatomical orientation comparison unit 16 can determine the relationship between each image part of the specific region and the anatomical orientation based on a change in the image characteristics for the lumen such as the large intestine.

Note that there are specific regions for which the relationship with the anatomical orientation cannot be determined from the image of the specific region. In this case, the anatomical orientation comparison unit 16 may obtain the relationship between each image part of the specific region and the anatomical orientation by inferring the relationship between the anatomical orientation and the specific region for which the relationship with the anatomical orientation cannot be determined, using information on the specific region for which the relationship with the anatomical orientation is determined.

A time-series image change determination unit 19 determines changes in the images obtained sequentially by inserting the endoscope, to determine a direction of movement of the endoscope distal end portion (insertion, withdrawal, or scanning observation of the specific region, or the like). The time-series image change determination unit 19 is a block configured by a circuit, software, an inference model, or the like, which can determine changes in time-series images and also determine a state of the imaging device approaching a specific diseased part, or the like.

In this way, in the examination in the lumen for observing a specific part in the human body, for example, it is possible, for example, using the same principle as when driving along a road and determining a current position based on specific scenery visible along the road, to grasp the position of the endoscope in the body and the relationship between the top and bottom of the image and a specific direction of the cross-section of the lumen at a specific position (although this is unlike the relationship between a road and a car running on the road with the orientation of the tires being fixed in the direction of gravity). When the image of the specific region is obtained, information on an inserted position based on image information of the image of the specific region can be acquired, and it is possible to align the relationship of the vertical directions of different images depending on whether the image of the specific region is detected in the vertical direction or the horizontal direction. With such a device, it is possible to confirm each specific region in the body in a manner like scanning, and it is also capable of performing a follow-up of the specific region in the body during another examination. In addition, it is possible to observe and record the specific region in the same way regardless of habits of the physician using the endoscope. Although the term “lumen” is used, in the case of the stomach, it is a bag rather than a tube, but the same concept can be applied. Although there are individual differences, the general shape of the bag is fixed, so it is possible to determine which region of the stomach the observation image is of. In the case of the large intestine, the shape of the tube may appear similar, making it unclear which region of the large intestine is being viewed, but if an insertion length of a scope can be detected, this can be used as a reference. To give an example using a car, this is similar to driving on a road with a monotonous scenery where it becomes difficult to determine the position. However, on highways, there are markers called “mile markers” that allow drivers to confirm their own position by the markers. Markings on a tube of the endoscope serve a similar function, so the markings are visually inspected to input information on the insertion length, or the markings are determined by photographing with a camera. Alternatively, such information is used to determine which part is observed.

The display control unit 17 can provide the endoscopic image subjected to image processing by the image processing unit 14 to the monitor 30 for display. In the present embodiment, in order to facilitate description of the image quality improvement for the specific region, which is a feature of the disclosure, an example where the tracking AF is performed is used, but the specific regions may be subjected to processing to improve the image quality, the visibility, and observability (as described above, to make it easier to use for observation and treatment of each region). At least one adjustment selected from focusing, exposure adjustment, illumination light control, and visibility enhancement is performed to obtain an image with excellent image quality and the like, and suitable for observation and treatment, on the display screen of the monitor 30. Note that it is not necessary to perform the tracking AF, and it is also possible to use the image of the part that is in focus as the image of the specific region for observation and treatment. In addition, the focus control is not essential, and a deep focus may be used.

Furthermore, the display control unit 17 can rotate and correct the image of the specific region and output the image to the monitor 30 so that the vertical and horizontal directions of the image of the specific region on the display screen are aligned based on the anatomical orientation. In other words, the display control unit 17 continuously rotates the endoscopic images sequentially obtained during insertion, to thereby display the endoscopic images subjected to processing in which the orientations of the endoscopic images sequentially obtained are unified based on the anatomical orientation (hereinafter, referred to as direction standardization processing). As a result, the specific region is always displayed in the same orientation on the screen, which is more suitable for observation and treatment. This is also one of processes for improving the image quality, the visibility, and the observability of the specific region, and is an example of what is described above as making it easier to use for observation and treatment of each region. Here, this processing not only makes a single image easy to see, but also makes the anteroposterior relationship between a plurality of temporally continuous images easy to understand by focusing on the continuity of the images, resulting in more advanced processing that can be called specific region observation continuous image processing.

The recording control unit 18 can provide the endoscopic image processed by the image processing unit 14 to the recording unit 40 for recording. The recording unit 40 is a recording device for recording on a predetermined recording medium such as a hard disk or a memory medium. In addition, the recording control unit 18 can provide the image displayed by the display control unit 17 to the recording unit 40 for recording. Therefore, observation and treatment of the specific region can be easily performed using the images recorded in the recording unit 40. In addition, a knowledge database is provided in the recording unit 40 so that reference information can be recorded for determining the images.

For the specific regions, the endoscopic images acquired during insertion are subjected to the processing such as the AF control or the image quality improvement, so that the images are suitable for observation and treatment. Therefore, the control unit 11 may detect a lesion part and the like by the image analysis on the endoscopic images acquired during insertion, and record in the recording unit 40 the endoscopic images with markings indicating the detection of the lesion part. Note that the control unit 11 may detect a state where changes can be recognized compared to a normal state, such as redness or phlegm, for example, and apply markings, even if the control unit 11 cannot recognize the changes as a lesion part.

By applying the marking to the endoscopic images during insertion, that is, images of non-observation target regions, it becomes easier to focus on and observe the region with the marking during withdrawal of the insertion portion 21, or the like, thereby increasing the likelihood that distinguishing of the lesion part and the like in regions other than the observation target regions is performed. For example, in a case where the observation target region is the stomach, the physician typically does not focus on the pharynx. However, in the present embodiment, if there is a lesion part in the pharynx, since the endoscopic image of the pharynx acquired during insertion is applied with the markings, the physician is more likely to focus on and observe the pharynx, which is normally the non-observation target region, during withdrawal, or the like. Note that when the markings are applied to a predetermined region during insertion, the control unit 11 may perform predetermined recommendation display so that attention is paid to the region with the markings during withdrawal.

In addition, insertion of the endoscope may cause redness in predetermined regions such as the vocal cords, for example. In the present embodiment, since the image quality of the endoscopic image acquired during insertion is also good, image comparison with the endoscopic image acquired during withdrawal can be performed with relatively high accuracy. Therefore, by comparing the endoscopic images acquired during insertion and withdrawal, it is possible to determine whether redness has occurred in the vocal cords or the like due to the insertion of the insertion portion 21. By effectively utilizing such endoscope distal end portion moving process images, various types of evidence can be acquired. This is an important technology that can be utilized in medical treatment for reporting and investigating causes.

### Operation

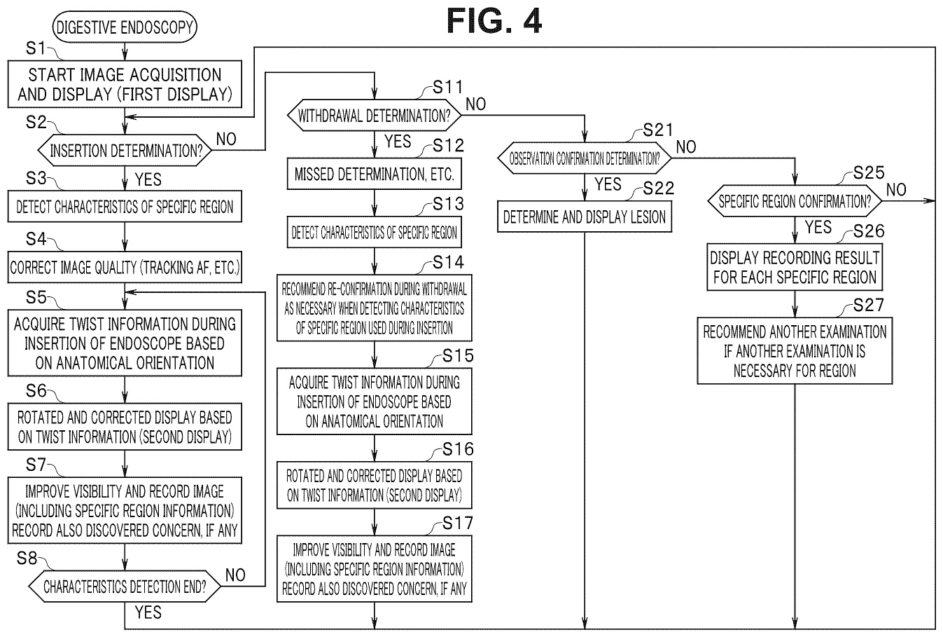

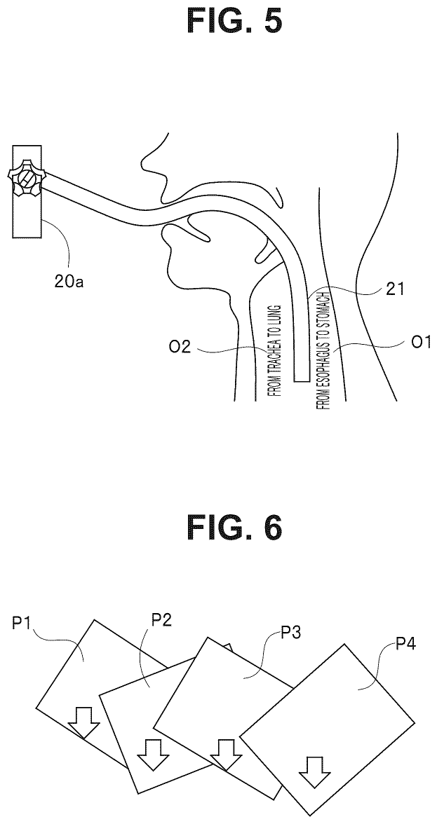

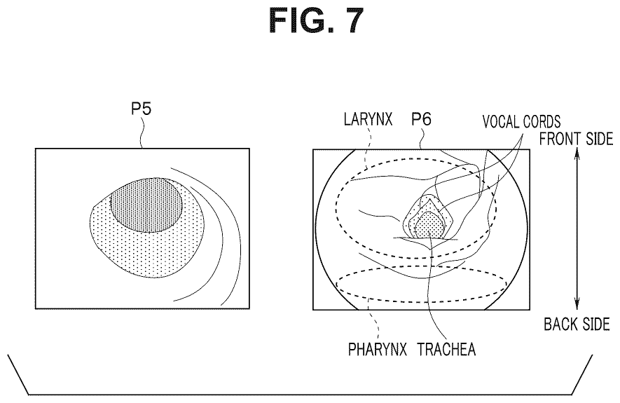

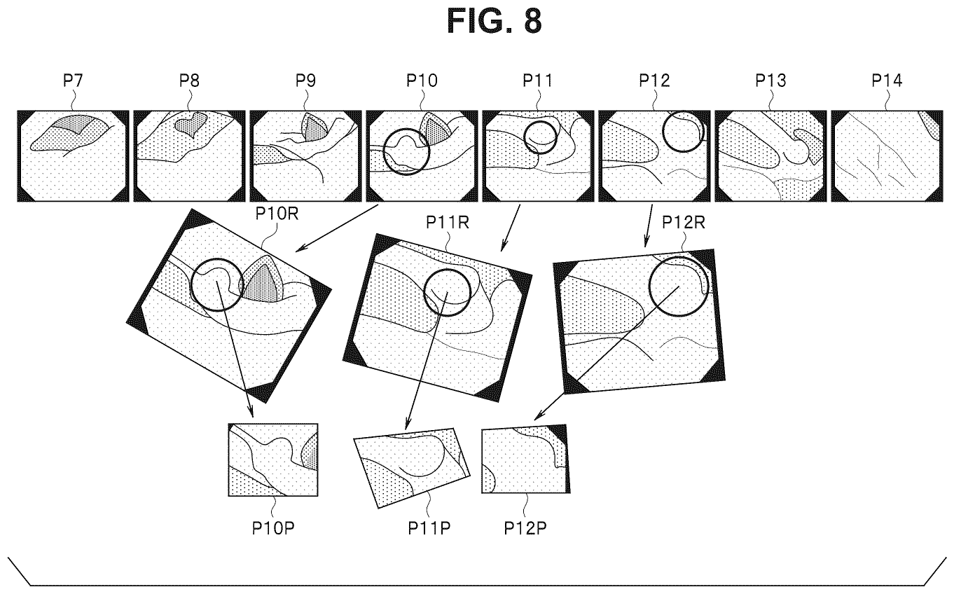

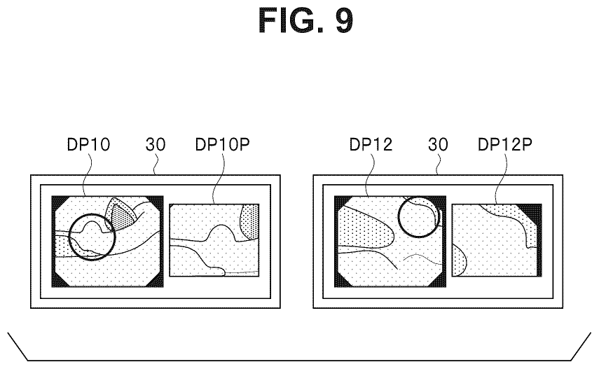

Next, operations of the embodiment thus configured will be described with reference to FIG. 4 to FIG. 8. FIG. 4 is a flowchart for explaining an operation of the first embodiment. FIG. 5 is an explanatory diagram for explaining insertion of the endoscope. FIG. 6 is an explanatory diagram showing an example of the relationship between the endoscopic image and the anatomical orientation. FIG. 7 is an explanatory diagram for explaining a method for determining the relationship between the specific region and the anatomical orientation. FIG. 8 is an explanatory diagram showing the endoscopic image that is acquired. FIG. 9 is an explanatory diagram showing a display example.

As shown in FIG. 5, for example, in the upper gastrointestinal endoscopy, the insertion portion 21 of the endoscope 20 is inserted through the mouth or the like. The physician advances and retracts the insertion portion 21 and, by operating an operation portion 20a constituting the endoscope 20, causes the bending portion at the distal end of the insertion portion 21 to bend, and twists the insertion portion 21, while advancing the insertion portion 21 from the esophagus toward the stomach. During insertion of the insertion portion 21, four endoscopic images P1 to P4 shown in FIG. 6, for example, are obtained. The arrows in the endoscopic images P1 to P4 indicate the same direction based on the anatomical orientation.

In the square frames in FIG. 6, the long sides show the horizontal direction of the imaging device, the short sides show the vertical direction of the imaging device, and the inclinations of the square frames respectively indicate orientations of the endoscopic images P1 to P4. The arrows in FIG. 6 indicate an example of changes in the relationship between the orientations of each of the endoscopic images and the anatomical orientation. The anatomical orientation comparison unit 16 obtains such a relationship between the orientations of each of the endoscopic images and the anatomical orientation.

In FIG. 7, the endoscopic image P5 shows an example where the lumen is the specific region, and the endoscopic image P6 shows an example where the specific regions are the pharynx and the larynx. The endoscopic image P5 shows the substantially straight lumen, and it is difficult to determine the relationship between the specific region and the anatomical orientation from the endoscopic image P5 alone. On the other hand, the endoscopic image P6 shows parts of the pharynx and the larynx. From the image characteristics, the pharynx portion is at the rear and the larynx is at the front, and the relationship between the endoscopic image P6 and the anatomical orientation is clear. The anatomical orientation comparison unit 16 obtains the relationship between each specific region and the anatomical orientation from such image characteristics. In addition, the anatomical orientation comparison unit 16 can infer the relationship between the specific region and the anatomical orientation for the endoscopic image P5, from the obtained relationship with the anatomical orientation for the endoscopic image P6 and characteristics of time-series changes in the endoscopic images. Furthermore, in a case where the lumen is bent, or the like, the anatomical orientation comparison unit 16 can obtain the relationship between the specific region and the anatomical orientation from characteristics of changes in the endoscopic images due to bending.

FIG. 4 shows a flow in a digestive endoscopy. In S1 of FIG. 4, image acquisition is started, and the acquired endoscopic image is displayed on the display screen of the monitor 30 (first display). The acquired image may be recorded in the recording unit 40 at this timing. In the present embodiment, such recorded images can be used for processing in later steps, and can generally be used as evidence and reporting for the medical treatment. The endoscopic image acquired by the image pickup device 22 is taken into the image processing device 10 by the image acquisition unit 13, and is subjected to predetermined signal processing by the image processing unit 14. The display control unit 17 provides the endoscopic image subjected to the signal processing to the monitor 30 for display.

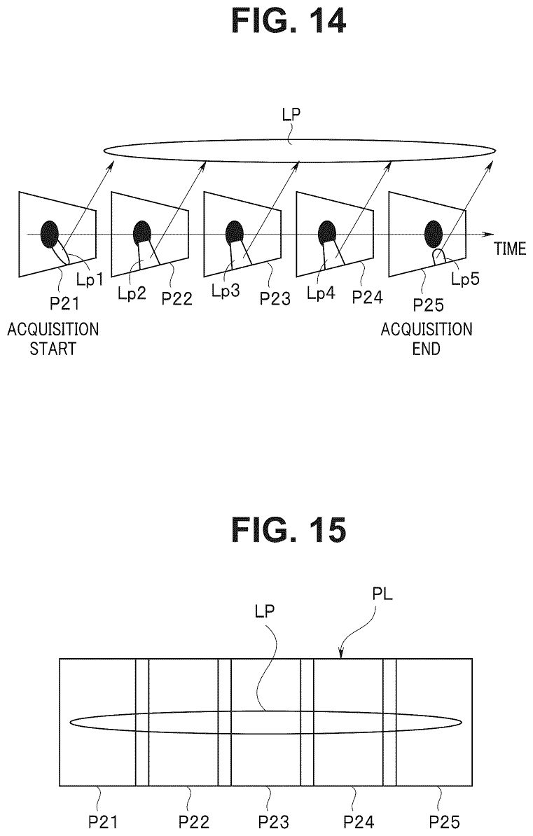

The control unit 11 determines whether the insertion portion 21 is inserted through the mouth, nose, or the like by image analysis (S2). This can be determined by detecting changes in the images having the characteristics of the position change progress image of the endoscope distal end portion. In other words, in the case of the lumen, an image of the deep lumen (deep portion (the back in a lumen length direction)) where illumination light from the endoscope distal end portion cannot reach has a black central portion (see FIG. 14) corresponding to the cross-section shape of the lumen (which is often substantially circular), and the insertion portion 21 advances toward that portion. Therefore, it may be determined that the periphery of the black circular portion gradually becomes brighter and the image changes so that an image pattern flows radially from there to the periphery of the screen. This determination is made by the time-series image change determination unit 19.

When insertion of the insertion portion 21 is started (YES in S2), the specific region determination unit 15 determines the image characteristics to detect the specific region (S3). The determination of the specific region may be made by referring to characteristic colors and shapes of the region, patterns such as blood vessels visible on the surface, or the like in a database provided in the recording unit 40. In addition, there is also a method for determining the specific region by receiving a transmission result of a magnetic transmitter or the like provided at the distal end of the endoscope by an external receiver, and determining the position of the endoscope distal end portion in the body. Furthermore, the specific region may be determined by using an inference model that has been trained using images of each region as training data, in addition to referring to a knowledge database or the like.

The image pickup control unit 12 controls the endoscope 20 so as to perform the tracking AF for the specific region in S4. In addition, the image processing unit 14 performs predetermined image signal processing so as to improve the image quality of the specific region (S4). In this way, images of the specific region that have excellent visibility and are suitable for observation and treatment can be obtained.

In addition, the anatomical orientation comparison unit 16 obtains the positional relationship (relationship of orientations) between the specific region in the image and the anatomical orientation (S5). For example, the anatomical orientation comparison unit 16 makes determination by acquiring the twist information during the insertion of the insertion portion 21.

The display control unit 17 rotates and corrects the endoscopic image based on the detection result (twist information) of the anatomical orientation comparison unit 16, and then outputs the endoscopic image to the monitor 30 (S6). As a result, the endoscopic image in which the directional relationship between the specific region and the anatomical orientation is maintained constant is displayed on the display screen of the monitor 30 (second display).

Processing circuitry of a system including a receiving device configured to receive temporally continuous image data (endoscopic images) from the endoscope configured to acquire images by the imaging device disposed at the distal end of the insertion portion performs such rotation and correction image processing and display of results thereof. Note that the receiving device may be constituted by the image acquisition unit 13. Here, since the specific region is detected based on the image characteristics in the images of the above described endoscopic image data, the endoscopic images temporally and continuously obtained are rotated and corrected respectively with reference to those.

FIG. 8 shows a series of endoscopic images P7 to P14 obtained during insertion, and shows each frame of a moving image obtained by image pickup, for example. The circles in the endoscopic images P10 to P12 in FIG. 8 show examples of the same specific region detected from the images. The specific region determination unit 15 detects the specific region surrounded by the circle in the endoscopic image P10 picked up and acquired. Based on the detection result of the specific region determination unit 15, the image pickup control unit 12 performs the tracking AF, and as a result, images of the same specific region are picked up in the endoscopic images P11 and P12 in focus. The images of the specific region are picked up with proper exposure, and high image quality processing is performed by the image processing unit 14.

However, when the endoscopic images P10 to P12 are acquired, the insertion portion 21 is twisted, and as shown by the inclinations of endoscopic images P10R to P12R, the endoscopic images P10 to P12 are rotated at different angles relative to each other based on the anatomical orientation. The anatomical orientation comparison unit 16 obtains the positional relationship (twist information) between the specific region and the anatomical orientation in each of the endoscopic images P10 to P12. The display control unit 17 causes the monitor 30 to display endoscopic images P10P to P12P, which are obtained by rotating the endoscopic images P10 to P12 based on the twist information, and by cropping and enlarging parts corresponding to the specific region. Note that an example of displaying the images after the cropping is shown, but it is also possible to perform only the rotating processing, as was done for the endoscopic images P10R to P12R.

FIG. 9 shows a display example in this case. The left side of FIG. 9 shows a display example of the monitor 30 when endoscopic image P10 is acquired, and the right side shows a display example of the monitor 30 when the endoscopic image P12 is acquired. On the left side of the display screen of the monitor 30, the endoscopic image DP10 or DP12 with improved image quality (first display) is displayed, and on the right side of the display screen, the endoscopic images DP10P or DP12P rotated and enlarged based on the anatomical orientation (second display) is displayed. The first display can be used to confirm an insertion direction and the like during insertion of the insertion portion 21, for example. In addition, the second display not only has a good image quality, but is also displayed so as to be aligned in an orientation based on the anatomical orientation, making it easy to use for observation, treatment, or the like. Note that the first display and the second display may be displayed to be aligned not only horizontally but also vertically.

The recording control unit 18 provides the endoscopic image to the recording unit 40 for recording (S7). In this case, the endoscopic image with visibility enhancement for the specific region is recorded. In addition, the recording control unit 18 records also specific region information related to the specific region. Furthermore, the recording control unit 18 also records information about any issues discovered during insertion.

The control unit 11 determines whether it is no longer possible to detect the characteristics of the specific region (characteristics detection end?) in S8. If the detection of the characteristics does not end (NO in S8), the processing returns to S5, and if the detection of the characteristics ends (YES in S8), the processing returns to S2. The specific region is determined by the specific region determination unit 15.

Note that in the above description, the example in which all the endoscopic images acquired during insertion of the endoscope 20 are used for both the first display and the second display has been described, but different frames may be set for frames used for the first display, frames used for the second display, or frames used for recording. In addition, although it is described that the tracking AF is performed on images after detecting the specific region, frames on which the tracking AF is performed and frames on which the tracking AF is not performed may be set. This inhibits the image portions related to insertion, which are used for confirming insertion, from being degraded due to the influence of the tracking AF.

When the control unit 11 determines that it is not in an insertion mode in S2 (NO in S2), the control unit 11 determines whether it is in a withdrawal mode in S11.

This can be determined by the time-series image change determination unit 19 detecting changes in images having the characteristics of the position change progress image of the endoscope distal end portion. In other words, in the case of the lumen, an image of the deep lumen (deep portion in the lumen length direction) where illumination light from the endoscope distal end portion cannot reach has a black central portion (see FIG. 14) corresponding to the cross-section shape of the lumen (which is often substantially circular), and the insertion portion 21 moves away from that portion. Therefore, it may be determined that the image changes so that the periphery of the black circular portion gradually becomes darker and the image changes so that the image pattern converges radially from the periphery of the screen.

When the control unit 11 determines that it is not in the withdrawal mode in S11 (NO in S11), the control unit 11 determines whether it is in an observation confirmation mode in S21.

This can be determined by the time-series image change determination unit 19 detecting changes in images having the characteristics of the observation image of the endoscope distal end portion. In other words, in the case of the lesion on the side surface of the lumen, an image of the deep lumen (deep portion in the lumen length direction) where illumination light from the endoscope distal end portion cannot reach during insertion and withdrawal has a black central portion (see FIG. 14) (detected in the image data) corresponding to the cross-section shape of the lumen (which is often substantially circular). From such a state, the distal end of the endoscope is bent toward a target wall surface. Therefore, it may be determined that a pattern of holes in the center moves toward the periphery of the screen, and then it may be determined that a region having a specific pattern is observed for a long time while moving closer or farther away or changing a viewing direction, to capture the same pattern such as blood vessels and irregularities in the lesion in the continuous images.

In the observation confirmation mode (YES in S21), the control unit 11 performs determination and discrimination of the lesion using the well-known image processing, AI (artificial intelligence) processing, or the like to display results thereof on the monitor 30 (S22).

In the withdrawal mode (YES in S11), the control unit 11 performs missed lesion determination or the like (S12). In the missed lesion determination, the control unit 11 determines whether there are any missed lesions in a case where the withdrawal is too fast or in parts that are likely to be a lesion part based on the image characteristics, or the like.

Next, the specific region determination unit 15 determines the image characteristics to detect a specific region (S13). The image characteristic determination analyzes patterns included in the image obtained from the imaging device in the distal end of the insertion portion of the endoscope to determine at which region in the human body the imaging device (the endoscope distal end portion) is located, and detects structures, colors, irregularities, blood vessel patterns, or the like, that are characteristic in that region. The specific region determination unit 15 uses the information such as the obtained patterns to determine a region with reference to a database in which images are associated with regions. The specific region determination unit 15 may be configured with an electronic circuit, and may be configured with a processor, a memory, or the like. In addition, the specific region determination unit 15 may use an inference model, and may use a model trained using images of the specific region annotated with information indicating the region, as training data. Furthermore, even if the region does not have the characteristics, it is possible to determine to which region an image corresponds, from the patterns of the before and after regions. For example, the region before and after a pattern specific to the vocal cords can be determined to be the entrance to the trachea or the esophagus. Furthermore, it is not necessary to use the endoscopic images, and the region may be determined by determining signals from a transmitter provided at the distal end of the endoscope with a sensor installed outside the body. If the examination is performed according to a specific time schedule, the region can be determined based on elapsed time information since the examination starts.

The control unit 11 uses the specific region information recorded during insertion, to recommend re-confirmation during withdrawal, as necessary (S14). Next, in S15 to S17, the same processing as in S5 to S7 during insertion is performed. In other words, also during withdrawal, the twist information is acquired, the endoscopic image is rotated based on the twist information, and the orientation of each endoscopic image is aligned based on the anatomical orientation to perform the second display. In addition, the images including the specific region information obtained during withdrawal are recorded. In this way, the temporally continuous image data (endoscopic images) are received from the endoscope that acquires the images by the imaging device provided at the distal end of the insertion portion, the specific region is detected based on the image characteristics in the images of the endoscopic image data, and the above temporally continuous endoscopic images are rotated and corrected based on a specific region image and recorded.

In addition, in S21, when the control unit 11 determines that it is not in the observation confirmation mode (NO in S21), the control unit 11 determines whether it is in a specific region confirmation mode in S25. The specific region confirmation mode is to confirm the recorded specific region information. The control unit 11 displays recording results for each specific region in the specific region confirmation mode (S26). If another examination is necessary for the specific region, the control unit 11 recommends the other examination (S27).

Note that FIG. 4 shows the example of performing the specific region confirmation mode during the endoscopy, but the specific region confirmation mode may be performed after the examination.

In the present embodiment, the processing of improving the image quality of the specific region in the human body in the image from the endoscopic images acquired by the endoscope during insertion of the endoscope is performed, and the relationship between the orientation of the specific region on the screen and the anatomical orientation is obtained, and the vertical and horizontal directions of the endoscopic image displayed on the screen is aligned based on the anatomical orientation. By the processing of standardizing the orientation of the image of the specific region based on the anatomical orientation (direction standardization processing), for example, the image part of the vocal cords in the larynx portion is standardized in the upward direction of the above endoscopic image, and the image part of the esophagus is standardized in the downward direction of the above endoscopic image. Since the direction standardization processing is performed, even endoscopic images during insertion of the endoscope have excellent visibility and are easy to use for observation, treatment, or the like.

In the above description, the example is shown in which the direction standardization processing during insertion of the endoscope is performed on the endoscopic image acquired during insertion of the endoscope, but the direction standardization processing may be performed at any timing, such as insertion or withdrawal of the endoscope, and after examination.

Furthermore, the example is described in which the direction standardization processing is performed on the endoscopic image acquired during insertion of the endoscope, but the direction standardization processing may be performed on the endoscopic image acquired during withdrawal of the endoscope. In the above example, the processing is classified into insertion, withdrawal, and observation confirmation, and the features of the present application are clearly explained. However, in the present disclosure, the processing does not have to be strictly classified into insertion, withdrawal, and observation confirmation, and the present disclosure actively acquires valuable information in any state and seeks to effectively utilize the acquired information, even if the acquired information is related to other medical departments.

### EXAMPLES OF SPECIFIC REGIONS

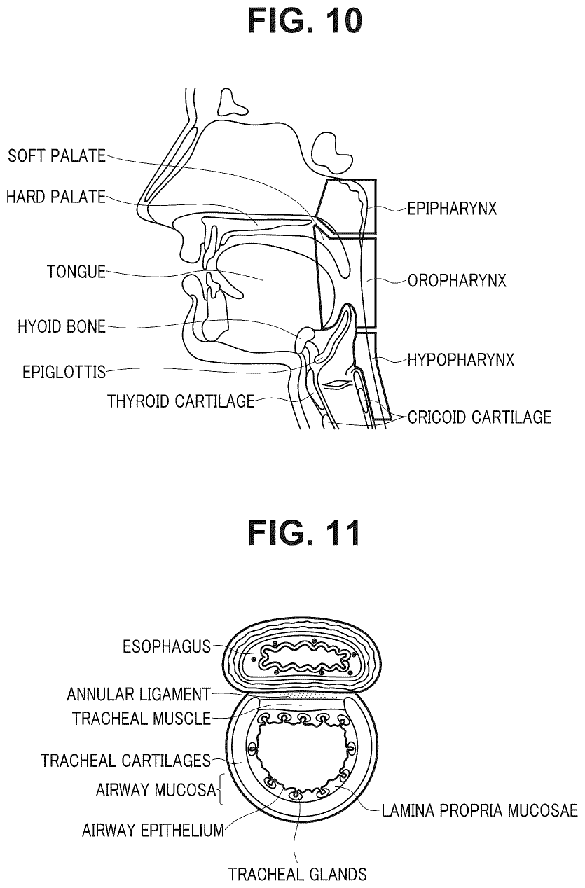

FIG. 10 to FIG. 12 are explanatory diagrams each showing an example of the specific regions.

FIG. 10 and FIG. 11 show examples of the specific regions that can be detected during insertion in the upper endoscopy. The example in FIG. 10 shows that the soft palate, the hard palate, the tongue, the hyoid bone, the epiglottis, the thyroid cartilage, the epipharynx, the oropharynx, the hypopharynx, the cricoid cartilage, and the like can be detected as specific regions. In addition, the example in FIG. 11 shows that the esophagus, the annular ligament, the tracheal muscle, the tracheal cartilages, the airway mucosa, the airway epithelium, the tracheal glands, and the lamina propria mucosae can be detected as specific regions.

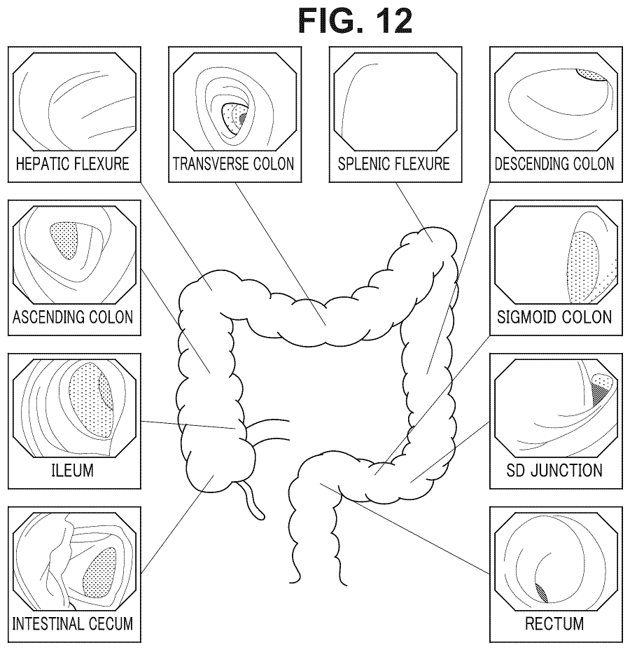

FIG. 12 shows an example of the specific regions that can be detected during insertion of the lower endoscopy, and picked-up images of respective specific regions. The example in FIG. 12 shows that the intestinal cecum, the ileum, the ascending colon, the hepatic flexure, the transverse colon, the splenic flexure, the descending colon, the sigmoid colon, the SD junction, and the rectum can be detected as specific regions. In addition, in the lower endoscopy, the anus, the dentate line and the like can also be detected as specific regions.

Note that not limited to the above examples, the specific region can be detected in any parts of the human body.

In the present embodiment, since the specific region other than the observation target region can be detected and the image of the specific region suitable for observation and treatment can be acquired during insertion of the endoscope, various diseases in each specific region other than the observation target region can be confirmed. For example, according to the present embodiment, during the upper gastrointestinal endoscopy, it is possible to confirm diseases such as epipharyngeal cancer, oropharyngeal cancer, hypopharyngeal cancer, acute epiglottitis, eosinophilic sinusitis, acute paranasal sinusitis, chronic sinusitis, allergic rhinitis, and the like, and during the lower gastrointestinal endoscopy, it is possible to confirm various diseases such as anal fissure, hemorrhoid, internal hemorrhoid, fistula, and the like.

### Second Embodiment

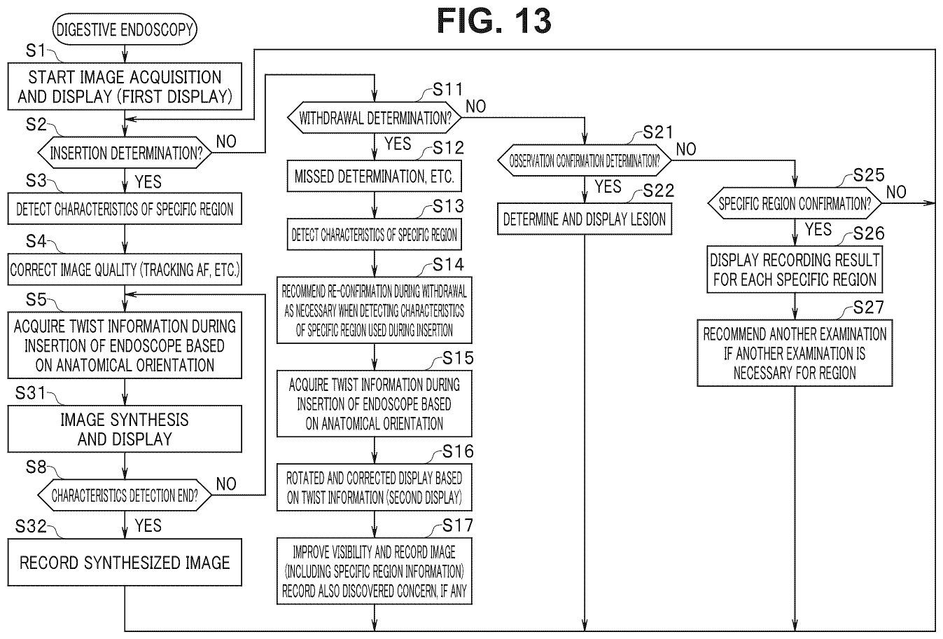

FIG. 13 is a flowchart showing an operation flow adopted in a second embodiment. In FIG. 13, the same procedures as in FIG. 4 are added with the same reference signs, and descriptions thereof are omitted. A hardware configuration in the present embodiment is the same as that in FIG. 1. In the present embodiment, the endoscopic images acquired during insertion are synthesized.

FIG. 13 differs from FIG. 4 in that S6 and S7 are omitted and S31 and S32 are added. After S5, the control unit 11 performs image synthesis in S31, and displays the generated synthesized image. The above acquired image may be recorded in the recording unit 40 at this timing. In the embodiment, such recorded images can be used for image synthesis or the like, and can also generally be used as evidence and reporting for the medical treatment. Next, the control unit 11 determines whether the detection of the characteristic ends in S8. If the detection of the characteristics ends (YES in S8), the recording control unit 18 provides the generated synthesized image to the recording unit 40 for recording.

FIG. 14 and FIG. 15 are explanatory diagrams for explaining the image synthesis in the second embodiment. The present embodiment relates to a system that not only rotates and corrects the images, but also synthesizes the corrected images to acquire new examination result images. The system includes a receiving device configured to receive temporally continuous image data (endoscopic images) from the endoscope configured to acquire the images by the imaging device disposed at the distal end of the insertion portion. The system may be a part of an in-hospital system for creating reports, or may be a part of an endoscope system that obtains examination images. The specific region is detected based on the image characteristics in the images of the endoscopic image data, the above temporally continuous endoscopic images are rotated and corrected respectively, based on the specific region image in the endoscopic image, and the rotated and corrected images are associated with text related to the symptoms of the above specific regions as examination results (reporting). Before the rotated and corrected images are used as the examination results, the rotated and corrected images are panoramically synthesized as shown in FIG. 15. Of course, if the regions of concern are within a single frame without synthesis, the panorama synthesis step is not necessary.

FIG. 14 shows endoscopic images P21 to P25 obtained by sequential image pickup along the time axis. The endoscopic images P21 to P25 include images of hole parts of the lumen shown in solid, and images Lp1 to Lp5 of the lesion parts occurring along the lumen. The images Lp1 to Lp5 are parts of one continuous lesion part LP, and are divided into frames and picked up by advancing the insertion portion 21 into the lumen while picking up images.

These images are not intended to pick up an image of the lesion part, but are assumed to be used as guide information during insertion, assuming that the examination target region is located deeper than the lumen. In other words, these images are images to be called the endoscope distal end portion moving process images or position change progress images. Note that if the lesion part is clearly visible as shown in the figure, the physician may notice the lesion part and begins observation, but in actual images, the lesion part may not be clearly visible to this extent. In the embodiment, it is assumed that the region is passed through without performing an examination unless the region is a present examination target.

In the present embodiment, as in the first embodiment, the twist information is acquired based on the anatomical orientation, and the endoscopic images P21 to P25 are subjected to the direction standardization processing based on the twist information so that the vertical and horizontal directions of the endoscopic images P21 to P25 are aligned based on the anatomical orientation. Therefore, as shown in FIG. 15, by superimposing the overlapped parts of adjacent frames of the endoscopic images P21 to P25 and panoramically synthesizing the endoscopic images P21 to P25, a synthesized image PL including the entirety of one lesion part LP can be obtained.

As above, in the present embodiment, since the orientation of each of the endoscopic images obtained continuously is aligned based on the anatomical orientation, the entirety of the lesion part having a relatively large size can be displayed. For example, the entirety of the lesion part and the like that are long in a length direction of the lumen (lumen direction) can be displayed, making it easier to recognize the entire lesion part. When the lesion that is not noticed in the image of each frame of the endoscope distal end portion moving process or the individual image of each frame of the position change progress is entirely displayed in this manner, the shape and boundaries of the lesion part are clearly shown so as to be easier to notice. For example, it becomes easier to make the determination based on the inference of the image determination. In addition, when passing through the specific region in the body, by performing focus control, exposure control, and other image processing control appropriate for that region, it may be easier to determine lesions or diseased parts more clearly. In the present embodiment, an image processing method is provided in which the specific region is detected based on the image characteristics of the endoscopic images acquired by the imaging device provided at the distal end of the insertion portion of the endoscope during insertion of the endoscope in the endoscopy, the directional relationship between the vertical and horizontal directions of the imaging device and the anatomical orientation of the human body into which the endoscope is inserted is determined based on the above endoscopic images, and the direction standardization processing that unifies the orientations of the above endoscopic images sequentially obtained based on the above anatomical orientation by continuously rotating the above endoscopic images for the above specific region sequentially obtained during the above insertion, based on the above directional relationship, is performed. Since the directional relationship with the anatomical orientation of the body is determined, it is also easier to organize the anatomical terms associated with the regions.

### Modification Example

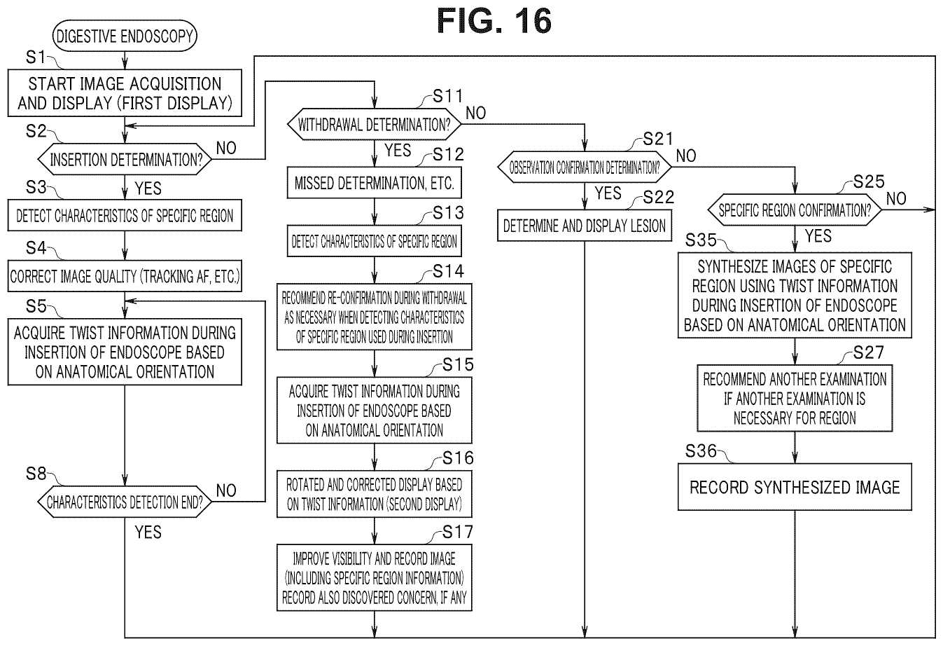

FIG. 16 is a flowchart showing a modification example of the second embodiment. In FIG. 16, the same procedures as in FIG. 13 are added with the same reference signs, and descriptions thereof are omitted. A hardware configuration in the present modification example is the same as that in FIG. 1. In the example in FIG. 13, the endoscopic images are synthesized during insertion of the endoscope, but in the present modification example, the endoscopic images are synthesized in the specific region confirmation mode after the endoscope is inserted.

In FIG. 16, the processing of S31 and S32 in FIG. 13 are omitted during insertion of the endoscope, and S35 corresponding to S31 is performed in the specific region confirmation mode (YES in S25). In addition, next to S27, S36 corresponding to S32 is performed. In other words, in the present modification example, generation and recording of the synthesized images are performed in the specific region confirmation mode.

Other configurations, operation, and effects are the same as those in the second embodiment.

Note that in FIG. 16, the example is shown in which the synthesis and recording of the endoscopic images are performed during the endoscopy, but the synthesis and recording of the endoscopic images may be performed after the examination. Focusing and illumination control with position control of the optical system is preferably performed during the examination, but adjustment of brightness of the screen with a gain, and adjustments such as color balance, contrast, gradation or the like may be performed on the image data recorded after the endoscopy.

### Third Embodiment

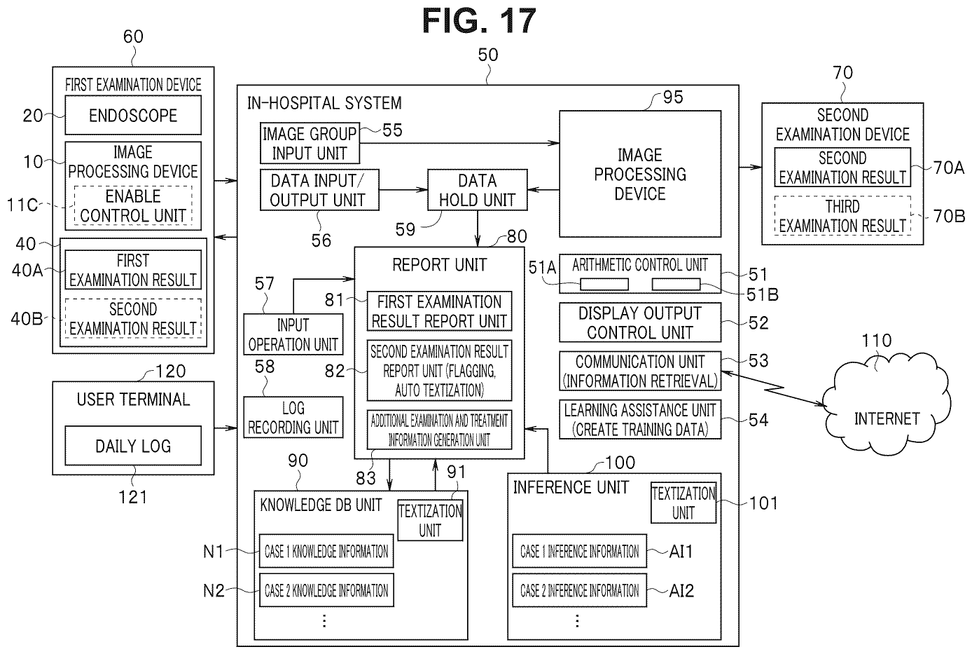

FIG. 17 is a block diagram showing a third embodiment. In FIG. 17, the same components as in FIG. 1 are added with the same reference signs, and descriptions thereof are omitted. The present embodiment shows an example in which the endoscopic images acquired during insertion of the endoscope are used after the examination.

A first examination device 60 in FIG. 17 corresponds to the endoscope 20, the image processing device 10, and the recording unit 40 in FIG. 1. The recording unit 40 stores a first examination result 40A and a second examination result 40B. The first examination result 40A is an examination result including the endoscopic images for the observation target regions. In other words, the first examination result 40A includes an examination result acquired by the physician with the intention to observe. The second examination result 40B is an examination result including the endoscopic images of the regions other than the observation target region (hereinafter, referred to as a non-observation target region) during insertion of the endoscope, in which the orientations of the endoscopic images are standardized based on the anatomical orientation. In other words, the second examination result 40B includes an examination result acquired without the intention of being observed by the physician. These examination results are transmitted to an in-hospital system 50. Of course, the second examination result 40B may be used to supplement the first examination result 40A, and may be obtained by recording information that cannot be recorded as the first examination result 40A, regardless of the intention of the physician.

An arithmetic control unit 51 of the in-hospital system 50 is configured to comprehensively control each part in the in-hospital system 50. Note that the arithmetic control unit 51 and each part in the in-hospital system 50 may be implemented by processing circuitry including one or more processors, such as a CPU or the like, may be configured to control each part by operating according to one or more programs stored in a memory, or may realize at least one of the functions with one or more hardware electronic circuits. For instance, the arithmetic control unit 51 may include processing circuitry including a CPU 51A and a memory 51B, and may be configured to take overall control of the in-hospital system 50 by the CPU 51A executing one or more programs stored in the memory 51B. Note that the in-hospital system 50 not only performs a medical care service such as examinations and treatments, but also handles various services within a hospital such as reception, prescriptions, accounting, and the like. However, FIG. 17 mainly shows the configuration related to diagnosis using the examination results.

A display output control unit 52 of the in-hospital system 50 controls various image display, printing, and data output. A communication unit 53 is capable of communicating with external lines such as Internet 110 and can acquire information from the Internet 110 or the like to enable information retrieval. A learning assistance unit 54 creates training data and the like for constructing an AI inference model using information acquired from the Internet 110 or the like, as described later.

A data input/output unit 56 of the in-hospital system 50 captures the first examination result 40A and the second examination result 40B from the first examination device 60, and provides the first examination result 40A and the second examination result 40B to the data hold unit 59. The data hold unit 59 includes a predetermined recording medium, and is configured to store various data. The data input/output unit 56 and an image group input unit 55 constitute the receiving device.

As described above, the direction standardization processing can be performed after the endoscopy. The image group input unit 55 in the in-hospital system 50 receives the first examination result 40A and an examination result including the endoscopic images that are acquired during insertion of the endoscope in the first examination device 60 and not subjected to the direction standardization processing (hereinafter, referred to as uncorrected data corresponding to the second examination result 40B), and outputs the first examination result 40A and the uncorrected data corresponding to the second examination result 40B to an image processing device 95. The image processing device 95 as a second processor has the same function as that of the image processing device 10 as a first processor in the first examination device 60, and performs the direction standardization processing on the uncorrected data corresponding to the second examination result 40B captured by the image group input unit 55, and acquires the same data as the second examination result 40B. If the second examination result 40B is not obtained in the first examination device 60, the image processing device 95 provides the obtained second examination result 40B to the data hold unit 59 for storing.

In general, the physician does not pay attention to images related to departments other than the medical department of his/her own specialty. For example, if the endoscope 20 of the first examination device 60 is a digestive endoscope, a specialist of the gastroenterological medicine who pays attention to the first examination result 40A does not pay attention to the second examination result 40B including image parts such as bronchi and the like. In the present embodiment, by performing diagnosis using the endoscopic images during insertion of the endoscope, which are not generally used, information effective for diagnosis and treatment is obtained.

A report unit 80 in the in-hospital system 50 includes a first examination result report unit 81, a second examination result report unit 82, and an additional examination and treatment information generation unit 83. The report unit 80 generates a report based on the first examination result 40A and the second examination result 40B. The physician operates an input operation unit 57 to control the report unit 80. The first examination result report unit 81 of the report unit 80 reads the first examination result 40A from the data hold unit 59, and displays an examination result based on the first examination result 40A on a monitor (not shown). The physician refers to the display on the monitor to diagnose the observation target region, and creates a medical record including information such as the presence or absence of lesion part, a condition of the lesion part, treatment methods, and prescriptions. The first examination result report unit 81 records the medical record created by the physician as a first examination result report on a recording medium (not shown).

On the other hand, the physicians who request an examination by the first examination device 60 are often specialists in the observation target region, for example, and may not be specialists in non-observation target regions, so the physicians typically do not use the second examination result 40B.

In the present embodiment, the second examination result report unit 82 of the report unit 80 captures the second examination result 40B from the data hold unit 59, and automatically performs diagnosis using the second examination result 40B. The second examination result report unit 82 performs automatic textization in which text describing diagnosis contents such as the presence of a lesion part, for example, is automatically generated as a diagnosis result.

The second examination result report unit 82 may capture corresponding text by image retrieval using a database that includes prepared text corresponding to images of each specific case, or may capture text prepared for characteristics of the image if the image characteristics (such as shape, region, size or color difference of lesion) differ from those of prepared images in the database. In addition to the database retrieval, the second examination result report unit 82 inputs images into an inference model that has been trained using training data created by annotating images with information capable of textization that can be read from the images and causes the inference model to output text.

In addition, the second examination result report unit 82 performs flagging in which various flags are generated corresponding to images of the detected lesion part, or the like. Note that the flag indicates the presence or absence of abnormalities, positional information of the lesion part, or the like, and examples of flags include various flags such as an abnormality flag indicating some kind of abnormality, a lesion flag indicating a lesion part, and a re-examination flag recommending re-examination, for example. The second examination result report unit 82 records the text and the flags as a second examination result report on a recording medium (not shown).

For example, suppose that the observation target region is the digestive organ, and the specific region that is the non-observation target region is the throat. For example, if there is a “red part” in the throat, the image of the specific region includes a red image part, and some kind of flag may be embedded in the second examination result 40B corresponding to the red image part. However, the physician of the digestive system who diagnoses the observation target region may be unable to distinguish the lesion part even by looking at the endoscopic image of the throat. To this, if the second examination result report unit 82 performs discrimination or the like of the lesion part for the red image part and determines that the red image part is a lesion part, the second examination result report unit 82 sets the abnormality flag, the lesion flag, the re-examination flag, or the like corresponding to the red image part, and generates text indicating that the image part is a lesion part, or the like. In addition, the second examination result report unit 82 may generate text or the like for recommending that a specialist physician perform a re-examination, as necessary.

Note that when infectious symptoms are determined from images of the throat, and the like, an instruction for immediate diagnosis, treatment, or the like may be generated.

The second examination result report unit 82 may perform flagging and automatic textization using a knowledge DB (database) unit 90 and an inference unit 100, for example.

The additional examination and treatment information generation unit 83 generates information that not only remains in the report but also encourages a patient to visit the hospital, such as prescriptions, appointments, and other recommendations for other medical examinations. This information may be generated by an input operation by the physician, or may be generated by the knowledge DB unit 90 or the inference unit 100.

The knowledge DB unit 90 stores examination data including case images for each of various cases, and knowledge information N1, N2, . . . (hereinafter, referred to as knowledge information N when there is no need to distinguish these pieces of information) which are medical knowledge such as causes of occurrence of cases, treatment methods for cases, and the like. The textization unit 91 of the knowledge DB unit 90 receives the second examination result 40B from the report unit 80, determines whether abnormalities such as lesion part or the like are included in the second examination result 40B by comparing the images included in the second examination result 40B with the knowledge information N1, N2, . . . of each cases, or the like, and generates a text explanation based on the knowledge information N regarding the presence or absence of abnormalities and their details.

In addition, the inference unit 100 stores inference information AI1, AI2, . . . (hereinafter, referred to as inference information AI when there is no need to distinguish these pieces of information) which are inference models such as disease names and causes of occurrence of cases, treatment methods for cases, and the like corresponding to the examination data including the case images for each of various cases, or the like. The textization unit 101 of the inference unit 100 receives the second examination result 40B from the report unit 80, performs inference using each piece of inference information for the examination data including the images included in the second examination result 40B, determines whether abnormalities such as lesion part or the like are included in the second examination result 40B, and generates a text explanation based on the inference information AI regarding the presence or absence of abnormalities and their details.

The learning assistance unit 54 uses information from the Internet 110 or the like to supplement the knowledge information N in the knowledge DB unit 90 and the inference information AI in the inference unit 100.

A second examination device 70 includes an endoscope and an image processing device, which are not shown in the drawing, in the same manner as the first examination device 60. The image processing device of the second examination device 70 has a function similar to that of the image processing device 10 of the first examination device 60. The endoscope of the second examination device 70 is different from the endoscope 20 of the first examination device 60, and for example, if the endoscope 20 of the first examination device 60 is a gastrointestinal endoscope, the endoscope of the second examination device 70 is a bronchial endoscope, or the like. In this case, the second examination device 70 acquires a second examination result 70A similar to the second examination result 40B, and also obtains a third examination result 70B different from the second examination result 70A during insertion of the endoscope.

In addition, the in-hospital system 50 can cooperate with a user terminal 120, and includes a log recording unit 58 that records a daily log 121 acquired by the user terminal 120. The user terminal 120 includes various sensors as necessary, and creates the daily log 121 containing health-related information for a user carrying the user terminal 120, such as an exercise time, a pulse rate, blood pressure, body temperature, a heart rate, a sleep time, a restroom usage time, and an electrocardiogram.

The daily log 121 may include or does not have to include information referred to as biological information or vital information. In some cases, specialized sensors are required to acquire the vital information, and such sensors may be separate devices from the user terminal 120, but the information acquired by these sensors is linked. The in-hospital system 50 can also acquire data obtained from such devices through the user terminal 120. This data may be recorded once in a recording unit in the user terminal 120.

The user terminal 120 sends the daily log 121 through the communication unit 53 and the like and provides the daily log 121 to the log recording unit 58. The log recording unit 58 records the daily log 121 for each user. On the other hand, the in-hospital system 50 includes a patient database (not shown) that stores history information of symptoms or the like for users whose daily logs 121 are acquired by the user terminal 120. The learning assistance unit 54 can supplement the knowledge information N in the knowledge DB unit 90 and the inference information AI in the inference unit 100 by training with the daily log 121 for a predetermined user recorded in the log recording unit 58, the history of symptoms or the like of the user, and information from the Internet 110, to improve accuracy.

In other words, logs and histories of cases or the like of a plurality of users (various patients) are included in the in-hospital system 50 and the Internet 110, and detailed analysis is possible by selecting and using information on patients having profiles similar to those of the user and indicating similar daily logs. When there is patient information with similar symptoms, treatment outcomes of patients with similar profiles and lifestyle patterns are more useful than those of other patients with different ages, genders, and lifestyle patterns. By grouping (categorizing) similar user groups in consideration of genetic information or the like, comparing the information, and inferring by AI, accuracy is further improved.

In the above description, it is described that the first examination device 60 unconditionally performs the direction standardization processing to create the second examination result 40B in which the orientations of the images of the specific region are standardized based on the anatomical orientation, but an enable control unit 11C configured by the control unit 11 in the image processing device 10 may be configured to control whether the direction standardization processing is performed on the endoscopic images acquired during insertion of the endoscope. In this case, it is possible to configure so as to control the enable control unit 11C based on the contents of the daily log 121 read from the log recording unit 58 by the knowledge DB 90 or the inference unit 100. By the enable control unit 11C, observation is possible in an image representation that is required for each situation, or in an image representation that is tailored to preferences of medical professionals such as the physician or the ease of understanding when a patient confirms the images.

In addition, the additional examination and treatment information generation unit 83 may determine the necessity of a re-examination or other examinations, treatment, or the like, not only based on the report created by the second examination result report unit 82 but also considering the contents of the daily log 121 recorded in the log recording unit 58.

The user terminal 120 is assumed to be a device that the user uses on a daily basis, and by acquiring information from the device as logs, it is possible to monitor the condition of the user on a daily basis. The additional examination and treatment information generation unit 83 determines whether the obtained data is normal, and when determining that the data deviates abnormally according to time changes, the additional examination and treatment information generation unit 83 can recommend the next medical treatment or create a reminder.

Even with a simple pedometer function, if a user who typically walks 10,000 steps per day suddenly walks only 1,000 steps on a given day, it may indicate a change in the lifestyle of the user. By determining such situations, information regarding what happened before that day, or the like can be retrieved from the daily log 121 or information in the medical records or the like, and utilized as medical information. For example, if there is a record of advice from a medical institution to rest, the relationship between the advice and the change in the lifestyle can be compared. If the change is due to the advice received, the medical institution can commend the patient for following the advice, thereby motivating the user to maintain health.

In addition to the pedometer, for example, it is also possible to detect breathing sounds of the user from audio collected by a microphone of a smartphone, compare the breathing sounds with normal breathing sound waveforms, and suspect respiratory diseases.

Abnormal breathing sounds include intermittent adventitious sounds, continuous adventitious sounds, pleural friction rubs, and the like, and depending on the user's disease, characteristic sound waveforms such as specific frequency components and repetition patterns can be obtained. In addition, it is also possible to determine a respiratory rate, which is considered normal at 12 to 18 breaths per minute. If within the normal range, there is no need for active cooperation with the in-hospital system, but in a case where other diseases are present, information such as the absence of symptoms related to breathing may be important for diagnosis and treatment. If there are abnormalities related to breathing, it is possible to branch to a form of medical treatment that is in accordance with the condition of the user and does not burden the user, by the following decisions.
Decision 1: Check the throat as well in the endoscopy in the next medical examination.
Decision 2: Recommend immediate visit to a doctor.
Decision 3: Consult a doctor during the next opportunity to visit a doctor.

Note that intracorporeal sounds such as heart sounds, arterial sounds, and bowel sounds, other than lung sounds, can be applied.

### Operation

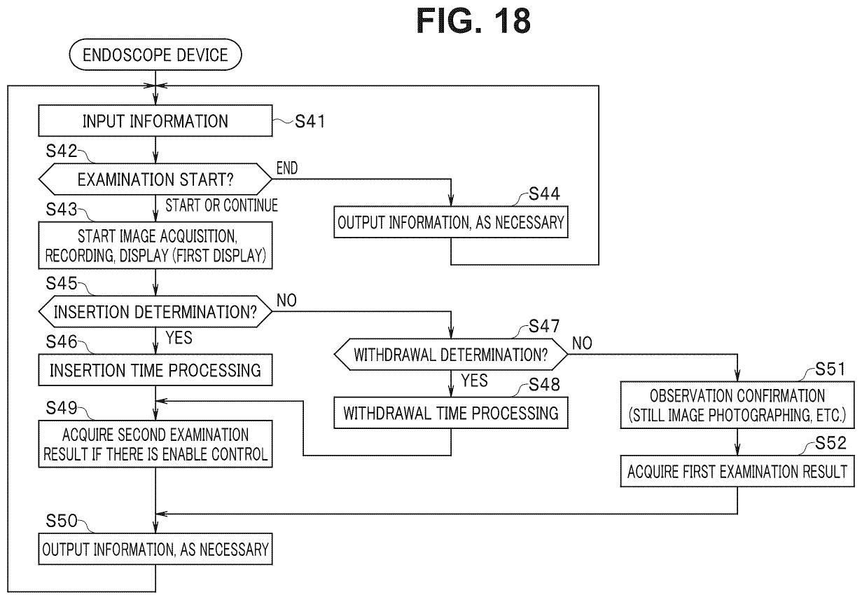

Next, operations of the embodiments thus configured will be described with reference to FIG. 18 to FIG. 20. FIG. 18 to FIG. 20 are flowcharts each explaining an operation of the third embodiment.

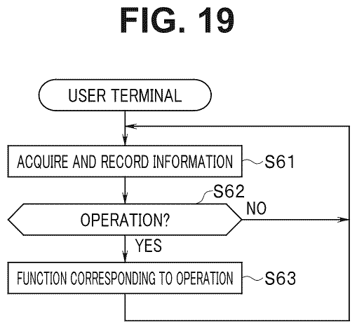

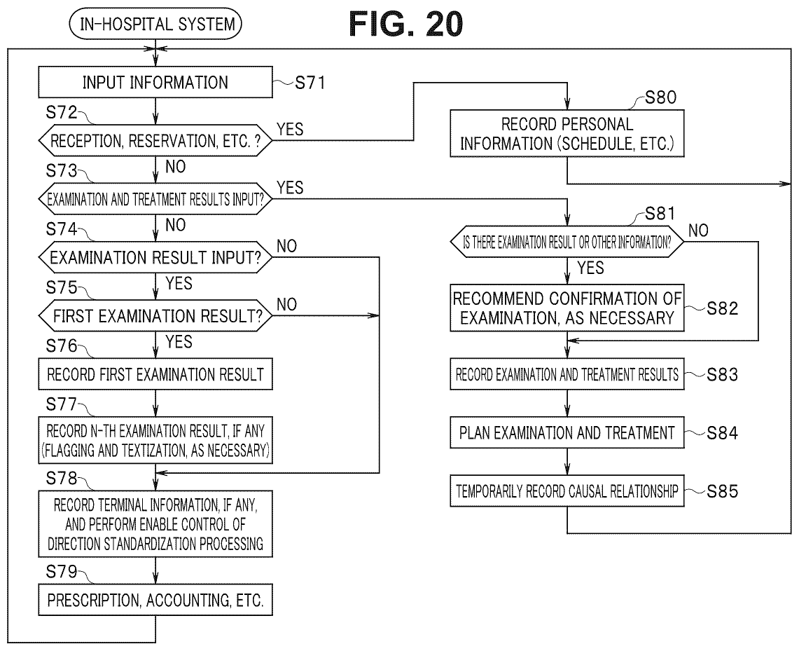

FIG. 18 to FIG. 20 show examples in which the in-hospital system 50, the first examination device 60, and the user terminal 120 operate in cooperation with one another. FIG. 18 shows an operation flow of the first examination device 60, which is an endoscope apparatus, FIG. 19 shows an operation flow of the user terminal 120, and FIG. 20 shows an operation flow of the in-hospital system 50.

In S41 of FIG. 18, information such as patient information on a patient undergoing an endoscopy, and the like are inputted by a user operation of an input operation portion (not shown) provided in the first examination device 60. The patient information may be already recorded in the patient database in the in-hospital system 50, and the control unit 11 may read the patient information from the patient database. The inputted information is recorded in the recording unit 40. In addition, the recorded patient database is updated with information about the examination to be undergone.

In S42, the control unit 11 determines whether the examination is started. When the examination is started (YES in S42), image acquisition is started (S43). The endoscopic image acquired by the image pickup device 22 is captured in the image processing device 10 by the image acquisition unit 13, and is subjected to predetermined signal processing by the image processing unit 14. The display control unit 17 provides the signal-processed endoscopic image to the monitor 30 for display. In addition, the recording control unit 18 provides the signal-processed endoscopic image to the recording unit 40 for recording.

Next, the control unit 11 determines whether the insertion portion 21 is inserted through the mouth, the nose, or the like, by image analysis (S45). When insertion of the insertion portion 21 is started (YES in S45), the control unit 11 performs insertion time processing (S46). If the control unit 11 determines that it is not in the insertion mode in S45 (NO in S45), the control unit 11 determines whether it is in the withdrawal mode in S47. If it is in the withdrawal mode (YES in S47), the control unit 11 performs withdrawal time processing (S48).

If the control unit 11 determines that it is not in the withdrawal mode in S47 (NO in S47), the observation confirmation mode is performed in S51. In the observation confirmation mode, still image photographing or the like is performed, for example. Next, the control unit 11 acquires the first examination result 40A (S52).

After the insertion time processing in S46 and the withdrawal time processing in S48, if there is enable control, the control unit 11 acquires the second examination result 40B (S49). In next S50, the control unit 11 performs information output, as necessary.

The image processing device 95 is provided in the in-hospital system 50, and the image processing device 95 can also acquire the second examination result 40B. For example, if the first examination device 60 does not create the second examination result 40B, the control unit 11 may sequentially transmit the first examination result 40A and the uncorrected data corresponding to the second examination result 40B to the in-hospital system 50 during the endoscopy (S50). In addition, the control unit 11 may transmit the first examination result 40A and the second examination result 40B, or the first examination result 40A and the uncorrected data corresponding to the second examination result 40B to the in-hospital system 50 in batch after the examination. In this case, the control unit 11 performs information output according to the NO determination in S42 (S44).

In this way, the first examination device 60 may create the first examination result 40A and the second examination result 40B, the first examination device 60 may output the first examination result 40A and the uncorrected data corresponding to the second examination result 40B, and the second examination result 40B may be obtained in the in-hospital system 50.

In S61 of FIG. 19, the user terminal 120 acquires information by various sensors, and sequentially records the acquired information in a memory (not shown) in the user terminal 120. By this, the daily log 121 is created. The user terminal 120 determines an operation in S62. When an operation occurs (YES in S62), the user terminal 120 performs a function corresponding to the operation (S63). For example, when the user terminal 120 has a telephone call function, a telephone call operation may be performed. When no operation is performed (NO in S62), information recording is continued. Note that when there is an operation to request transmission of the daily log 121 from the in-hospital system 50, the accumulated daily log 121 is transmitted to the in-hospital system 50 in response to the request (S63).

In S71 of FIG. 20, various types of information are inputted by a user operation of the input operation unit (not shown). The arithmetic control unit 51 determines whether the inputted information is information related to reception, reservation, or the like (S72). If the inputted information is the information related to reception, reservation, or the like (YES in S72), the arithmetic control unit 51 records personal information such as schedules in the patient database in S80, and returns to the processing of S71.

In a case of NO determination in S72, the arithmetic control unit 51 determines whether the inputted information is information related to examination and treatment results (S73). In a case of NO determination in S73, the arithmetic control unit 51 determines whether the inputted information is information related to an examination result (S74). If the inputted information is the information related to the examination result (YES in S74), the arithmetic control unit 51 determines whether the inputted information is information related to the first examination result 40A (S75). If the inputted information is the information related to the first examination result 40A (YES in S75), the arithmetic control unit 51 records the first examination result 40A in the data hold unit 59 (S76). In addition, if there is the second examination result 40B, 70A or the third examination result 70B (n-th examination result) in S77, the arithmetic control unit 51 records the examination result in the data hold unit 59. The second examination result 40B, 70A and the third examination result 70B are flagged and converted into text, as necessary.

If there is the daily log 121 (terminal information) from the user terminal 120, the arithmetic control unit 51 records the information in the log recording unit 58. After the processing of S77 ends, or in a case of NO determination in S74 or S75, the arithmetic control unit 51 performs the enable control of the direction standardization processing based on the daily log 121 (S78). The arithmetic control unit 51 performs prescription, accounting, and the like in S79, and returns to the processing of S71.

If the inputted information is information related to the examination and treatment results (YES in S73), the arithmetic control unit 51 determines whether there is the examination result or other information in S81. In S81, for example, it is determined that the endoscopic images or X-ray images exist. If the endoscopic images or the X-ray images exist, the arithmetic control unit 51 recommends that the physician confirm the examination result as necessary (S82), and if the endoscopic images or the X-ray images do not exist, the processing proceeds to S83.

In order to verify hypotheses regarding how the conditions of specific types of patients change over specific periods, research that collects new cases in the future and “verifies” the new cases is called “prospective research”, and research that collects past cases and “verifies” the past cases is called “retrospective research”. For such verification, it is necessary to track changes in a health status of a specific patient. In addition, research by tracking changes in the health statuses of a group of patients with specific conditions and with specific profiles may be conducted. In the present embodiment, since the daily log and the examination results in the hospital are linked, it is possible to create an environment that facilitates such research.

For example, when a plurality of pieces of patient information with similar profiles or histories (determined by the accumulation of the daily log) are collected, the collected patient information may be organized by tagging and grouping. For example, if patient data of patients in their fifties is initially collected, and many patients with similar symptoms are examined, it is possible to create new groups, by such as classifying of male and female patients into separate categories. The creation of new groups with a new angle or an increase in the number of group members is referred to as “updating” of the group, and in this case, more detailed findings can be obtained as knowledge.

When determining a treatment plan by predicting how the patient's current condition will develop, it is also possible to recommend papers (the paper authors themselves—or, alternatively, the researchers, universities, hospitals, or institutions to be contacted) or the like that are appropriate for the profile, a disease name, and symptoms of the patient. In particular, if the medical image data is subjected to the direction standardization processing considering the anatomical orientation, which is the feature of the present application, or if the medical image data is organized based on the anatomical characteristics, it is possible to easily make a correspondence with other research using region names included in the anatomical terms. When such regions serve as keywords during classification, it is also possible to link information other than the images.

Of course, when starting the prospective research, images organized by region information or the direction standardization processing are easy to compare with data of other patients or from other medical institutions. In addition, when such classifications are organized, it is possible to determine which cases are rare, for example, by determining that cases cannot be included in the already determined classification, or by determining that there is no data that can be entered in specific rows or columns when creating a table of cases and classifications. In other words, it is possible to determine the rarity of the examination results. The rare cases are difficult to collect even with conscious effort, so it is possible to effectively utilize data of a patient who happens to visit for examination in research with the systems and method of the present application.

In S83, the arithmetic control unit 51 records the results of the examination and the treatment performed by the physician in the patient database. The arithmetic control unit 51 plans and presents the next examination and the treatment to the physician, and records the plan in the patient database (S84). In addition, the physician refers to the diagnosis result, papers and the like to obtain a causal relationship that becomes the knowledge information of the cases. The in-hospital system retrieves the examination result held by the in-hospital system, papers published outside the hospital, or the like, extracts information that can be determined to have a causal relationship, and displays the information to the physician through a display output control unit, and when the information determined to have the causal relationship is not extracted, the in-hospital system notifies the physician through the display output control unit that there is a possibility of a rare case, thereby promoting research. The arithmetic control unit 51 temporarily records the information of the causal relationship obtained by the physician in a memory (not shown) of the learning assistance unit 54, in order to accumulate the knowledge information N. The learning assistance unit 54 can update the knowledge information N based on the information of the causal relationship that is temporarily recorded.

When a plurality of pieces of patient information with similar profiles are collected, the collected patient information may be organized by tagging and grouping the patient information. When many patients with similar profiles are examined, the patients can be classified into different categories by gender or age groups. The creation of groups with a new angle or an increase in the number of members in a group is considered an “update” of the group, and in this case, the knowledge can obtain more detailed findings. With such devices, based on the information of groups of a plurality of patients with similar histories, it is possible to infer that people with such symptoms are likely to have this disease, or recommend retrospective research showing that people with such symptoms or lifestyles are likely to develop such diseases.

If the daily logs are insufficient, it is also possible to add information such as results of a medical interview, to add such information by the medical professional, or to add such information on the initiative of the patient and their relatives, by using a checklist in a questionnaire format on a screen of a smartphone with arranged items such as “drink alcohol” or “parent has . . . ”. Of course, it is also possible to build a data set automatically organized by AI from natural language obtained by textization of medical interview sheets or conversations, diagnostic image groups, logs acquired from daily life. By cooperation with a PC or the smartphone that is used by a user as a patient candidate on a daily basis, a process of the medical interview can be simplified, and additional information can be acquired in a “monitoring” manner.

As above, in the present embodiment, the endoscopic images acquired during insertion of the endoscope can be used after the examination. For the endoscopic images acquired during insertion of the endoscope, to which no attention is paid, because the endoscopic images are images of the non-observation target regions, textization of contents of the symptoms or the like can be automatically performed to generate a report from the endoscopic images. Therefore, even when the physician who is not a specialist in the non-observation target region performs diagnosis, or the like, the physician can easily notice the lesion part and the like, and easily make determination regarding treatment or re-examination.

As described above, in the present application, when the endoscope including the cylindrical distal end portion is inserted along the lumen, or the like, the vertical direction of the image tends to rotate clockwise or counterclockwise without its orientation being maintained, but by aligning the region using the anatomical characteristics of the observation regions and aligning the top and bottom of the images, it is possible to obtain images such as those published in papers or the like. The images with top and bottom adjusted in this way make it easier to detect characteristic findings, symptoms, or the like. In addition, by focusing, exposure control, and image processing to obtain such images, it is possible to photograph clear images. This facilitates diagnosis and improves determination accuracy. By presenting these images to patients and their families, there is an advantage in that it can be communicated visually in an easily understandable manner. This enhances patient acceptance and enables smoother examination. The images with the aligned top and bottom can be easily compared with past images, which is useful for monitoring the condition of the patient of a personal physician. Of course, the result of the medical interviews can also be referenced, and by linking with smartphones or the like in particular, the process of the medical interviews can be simplified, and the additional information can be acquired in a “monitoring” manner. In the embodiment, the features of the present application are clearly organized in an example in which the processing is divided into insertion, withdrawal, and observation, but the aim is to effectively utilize any valuable information obtained in any state, even if it is related to other medical departments.

The present disclosure is not limited to the above embodiments as they are, and in the implementation phase, the components may be modified and embodied within the scope not departing from the gist thereof. In addition, various disclosures can be formed by appropriately combining a plurality of components disclosed in each of the above embodiments. For example, some of all the components indicated in the embodiments may be deleted. Furthermore, components from different embodiments may be combined as appropriate.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), microcontrollers, digital signal processors (DSPs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, ROM, or other storage media) that stores data, software, firmware, or instructions contributing to the processing operations. Accordingly, the processing circuitry may be implemented as a specialized hardware circuit having fixed logic, a programmable circuit executing instructions stored in an internal or external memory, or any combination thereof. The processing circuitry may be configured as, or include, one or more processors. Thus, the term “processor” used in the description of embodiments is to be understood as a specific example of the processing circuitry or as a component included within the processing circuitry. Furthermore, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:
Clause 1: An endoscope apparatus comprising:
   an endoscope including an imaging device at a distal end of an insertion portion; and
   a processor, wherein
   the processor is configured to:
      based on image changes of a plurality of endoscopic images acquired by the imaging device during insertion of the endoscope in an endoscopy, determine the endoscopic images as non-observation target images or observation target images;
      detect a specific region based on image characteristics in the non-observation target images;
      determine images including the specific region among the non-observation target images, as correction target images;
      determine a directional relationship between imaging directions of the imaging device and an anatomical orientation of a human body into which the endoscope is inserted, based on the correction target images; and
      continuously rotate the correction target images that are sequentially obtained during the insertion based on the directional relationship, to perform direction standardization processing that unifies orientations of the correction target images that are sequentially obtained, based on the anatomical orientation.
Clause 2: The endoscope apparatus according to clause 1, wherein the processor is configured to display the correction target images before the direction standardization processing and the correction target images after the direction standardization processing side by side on a display screen.
Clause 3: The endoscope apparatus according to clause 1, wherein the processor is configured to detect a region in which anatomical characteristics of the human body can be detected based on the image characteristics in the correction target image, as the specific region.
Clause 4: The endoscope apparatus according to clause 1, wherein the processor is configured to detect a region in which anatomical characteristics of the human body can be inferred from a region in which the anatomical characteristics can be detected based on the image characteristics of the correction target images, as the specific region.
Clause 5: The endoscope apparatus according to clause 1, wherein the processor is configured to obtain the directional relationship based on a shape of a lumen obtained from the image characteristics in the correction target images.
Clause 6: The endoscope apparatus according to clause 1, wherein the processor is configured to obtain the directional relationship based on a change in the image characteristics in the correction target images.
Clause 7: The endoscope apparatus according to clause 1, wherein the processor is configured to perform cropping processing of a part corresponding to the specific region in the correction target images subjected to the direction standardization processing.
Clause 8: The endoscope apparatus according to clause 1, wherein the processor is configured to perform at least one image processing of focusing, exposure adjustment, illumination light control, or visibility enhancement, on a part corresponding to the specific region.
Clause 9: The endoscope apparatus according to clause 1, wherein the processor is configured so that, by the direction standardization processing, image parts of vocal cords in a larynx are standardized in an upward direction of the correction target images, and image parts of an esophagus are standardized in a downward direction of the correction target images.
Clause 10: An image processing device comprising a processor, wherein the processor is configured to:
   based on image changes of a plurality of endoscopic images acquired by an imaging device provided at a distal end of an insertion portion of an endoscope during insertion of the endoscope in an endoscopy, determine the endoscopic images as non-observation target images or observation target images;
   detect a specific region based on image characteristics in the non-observation target images;
   determine images including the specific region among the non-observation target images, as correction target images;
   determine a directional relationship between imaging directions of the imaging device and an anatomical orientation of a human body into which the endoscope is inserted, based on the correction target images; and
   continuously rotate the correction target images that are sequentially obtained during the insertion based on the directional relationship, to perform direction standardization processing that unifies orientations of the correction target images that are sequentially obtained, based on the anatomical orientation.
Clause 11: The image processing device according to clause 10, wherein the processor is configured to detect a region in which anatomical characteristics of the human body can be detected based on the image characteristics in the correction target images, as the specific region.
Clause 12: The image processing device according to clause 10, wherein the processor is configured to detect a region in which anatomical characteristics of the human body can be inferred from a region in which the anatomical characteristics of the human body can be detected based on the image characteristics in the correction target images, as the specific region.
Clause 13: The image processing device according to clause 10, wherein the processor is configured to synthesize a series of the correction target images subjected to the direction standardization processing.
Clause 14: An image processing method comprising:
   receiving data of temporally continuous endoscopic images including non-observation target images and observation target images from an endoscope that acquires images by an imaging device provided at a distal end of an insertion portion;
   detecting a specific region based on image characteristics in the non-observation target images;
   determining images including the specific region among the non-observation target images, as correction target images;
   rotating and correcting each of the temporally continuous correction target images based on an image of the specific region in the correction target images; and
   associating the image rotated and corrected with text related to a symptom of the specific region as an examination result.
Clause 15: An in-hospital system comprising:
   a receiving device configured to receive data of temporally continuous endoscopic images including non-observation target images and observation target images from an endoscope that acquires images by an imaging device provided at a distal end of an insertion portion; and
   a processor, wherein
   the processor is configured to:
      detect a specific region based on image characteristics in the non-observation target images;
      determine images including the specific region among the non-observation target images, as correction target images;
      rotate and correct each of the temporally continuous correction target images based on an image of the specific region in the correction target images; and
      associate the images rotated and corrected with text related to a symptom of the specific region as an examination result.
Clause 16: The in-hospital system according to clause 15, wherein the processor is configured to receive a daily log, which is health-related information of a user, from a user terminal configured to acquire and hold the daily log, and enable the rotation and the correction based on the daily log.
Clause 17: An image processing method comprising:
   based on image changes of a plurality of endoscopic images acquired by an imaging device provided at a distal end of an insertion portion of an endoscope during insertion of the endoscope in an endoscopy, determining the endoscopic images as non-observation target images or observation target images;
   detecting a specific region based on image characteristics in the non-observation target images;
   determining images including the specific region among the non-observation target images, as correction target images;
   determining a directional relationship between imaging directions of the imaging device and an anatomical orientation of a human body into which the endoscope is inserted, based on the correction target images; and
   continuously rotating the correction target images that are sequentially obtained during the insertion based on the directional relationship, to perform direction standardization processing that unifies orientations of the correction target images that are sequentially obtained, based on the anatomical orientation.
Clause 18: The image processing method according to clause 17, further comprising panoramically synthesizing a series of the correction target images subjected to the direction standardization processing.
Clause 19: An image processing program configured to cause a computer to execute a procedure comprising:
   based on image changes of a plurality of endoscopic images acquired by an imaging device provided at a distal end of an insertion portion of an endoscope during insertion of the endoscope in an endoscopy, determining the endoscopic images as non-observation target images or observation target images;
   detecting a specific region based on image characteristics in the non-observation target images;
   determining images including the specific region among the non-observation target images, as correction target images;
   determining a directional relationship between imaging directions of the imaging device and an anatomical orientation of a human body into which the endoscope is inserted, based on the correction target images; and
   continuously rotating the correction target images that are sequentially obtained during the insertion based on the directional relationship, to perform direction standardization processing that unifies orientations of the correction target images that are sequentially obtained, based on the anatomical orientation.

What is claimed is:

1. An endoscope apparatus comprising:
an endoscope including an imaging device disposed at a distal end of an insertion portion and configured to capture a plurality of endoscopic images when the endoscope is inserted into a human body; and
processing circuitry configured to:
based on image changes of the plurality of endoscopic images, determine whether each endoscopic image is a non-observation target image or an observation target image;
detect a specific region based on image characteristics in endoscopic images determined as the non-observation target images;
determine images including the specific region among the non-observation target images as correction target images;
determine a directional relationship between a direction of the imaging device and an anatomical orientation of the human body, based on the correction target images; and
perform direction standardization processing to successively rotate the correction target images obtained sequentially during the insertion of the endoscope based on the directional relationship, thereby standardizing orientations of the correction target images based on the anatomical orientation as a reference.

2. The endoscope apparatus according to claim 1,
wherein the processing circuitry is further configured to cause a display to display a first image and a second image side by side, the first image being one of the correction target images before the direction standardization processing, and the second image being the first image subjected to the direction standardization processing.

3. The endoscope apparatus according to claim 1,
wherein the processing circuitry is further configured to detect, as the specific region, a region in which anatomical characteristics of the human body are detectable based on the image characteristics in the non-observation target images.

4. The endoscope apparatus according to claim 1,
wherein the processing circuitry is further configured to detect, as the specific region, a region in which anatomical characteristics of the human body are inferable from another region in which the anatomical characteristics are detectable based on the image characteristics in the non-observation target images.

5. The endoscope apparatus according to claim 1,
wherein the processing circuitry is further configured to determine the directional relationship based on a shape of a lumen obtained from the image characteristics in the correction target images.

6. The endoscope apparatus according to claim 1,
wherein the processing circuitry is further configured to determine the directional relationship based on a change in the image characteristics in the correction target images.

7. The endoscope apparatus according to claim 1,
wherein the processing circuitry is further configured to crop an area corresponding to the specific region from the correction target images subjected to the direction standardization processing.

8. The endoscope apparatus according to claim 1,
wherein the processing circuitry is further configured to perform at least one adjustment selected from focusing, exposure adjustment, illumination light control, and visibility enhancement, for an area corresponding to the specific region.

9. The endoscope apparatus according to claim 1,
wherein the processing circuitry is further configured to orient, by the direction standardization processing, an image portion corresponding to vocal cords in an upward direction of each correction target image, and an image portion corresponding to an esophagus in a downward direction on each correction target image.

10. An image processing device comprising:
a connection interface; and
processing circuitry configured to:
receive, via the connection interface, a plurality of endoscopic images captured by an imaging device of an endoscope when the endoscope is inserted into a human body;
based on image changes of the plurality of endoscopic images, determine whether each endoscopic image is a non-observation target image or an observation target image;
detect a specific region based on image characteristics in endoscopic images determined as the non-observation target images;
determine images including the specific region among the non-observation target images as correction target images;
determine a directional relationship between a direction of the imaging device and an anatomical orientation of the human body, based on each correction target image; and
perform direction standardization processing to successively rotate the correction target images obtained sequentially during the insertion of the endoscope based on the directional relationship, thereby standardizing orientations of the correction target images based on the anatomical orientation as a reference.

11. The image processing device according to claim 10,
wherein the processing circuitry is further configured to detect, as the specific region, a region in which anatomical characteristics of the human body are detectable based on the image characteristics in the non-observation target images.

12. The image processing device according to claim 10,
wherein the processing circuitry is further configured to detect, as the specific region, a region in which anatomical characteristics of the human body are inferable from another region in which the anatomical characteristics of the human body are detectable based on the image characteristics in the non-observation target images.

13. The image processing device according to claim 10,
wherein the processing circuitry is further configured to synthesize a series of the correction target images subjected to the direction standardization processing.

14. The image processing device according to claim 10,
wherein the processing circuitry comprises:
a processor; and
a memory storing computer-readable instructions configured to, when executed by the processor, cause the processor to:
receive the plurality of endoscopic images;
based on the image changes of the plurality of endoscopic images, determine whether each endoscopic image is the non-observation target image or the observation target image;
detect the specific region based on the image characteristics in the non-observation target images;
determine the images including the specific region among the non-observation target images as the correction target images;
determine the directional relationship between the direction of the imaging device and the anatomical orientation of the human body, based on each correction target image; and
perform the direction standardization processing to standardize the orientations of the correction target images based on the anatomical orientation as the reference.

15. An in-hospital system comprising:
a receiving device configured to receive, from an endoscope, temporally continuous endoscopic images captured by an imaging device, the endoscopic images including non-observation target images and observation target images; and
processing circuitry configured to:
detect a specific region based on image characteristics in the non-observation target images;
determine images including the specific region among the non-observation target images as correction target images;
perform rotational correction on each of the temporally continuous correction target images based on the specific region in the correction target images; and
associate the images subjected to the rotational correction with text related to a symptom of the specific region as an examination result.

16. The in-hospital system according to claim 15,
wherein the processing circuitry is further configured to:
receive a daily log including information regarding health of a user, from a user terminal configured to acquire and store the daily log; and
enable the rotational correction based on the daily log.

17. An image processing method implementable by processing circuitry configured to be connected to an endoscope, the image processing method comprising:
receiving, from the endoscope, temporally continuous endoscopic images captured by an imaging device, the endoscopic images including non-observation target images and observation target images;
detecting a specific region based on image characteristics in the non-observation target images;
determining images including the specific region among the non-observation target images as correction target images;
performing rotational correction on each of the temporally continuous correction target images based on the specific region in each correction target image; and
associating the images subjected to the rotational correction with text related to a symptom of the specific region as an examination result.

18. An image processing method implementable by processing circuitry configured to be connected to an endoscope, the image processing method comprising:
receiving a plurality of endoscopic images captured by an imaging device of the endoscope when the endoscope is inserted into a human body;
based on image changes of the plurality of endoscopic images, determining whether each endoscopic image is a non-observation target image or an observation target image;
detecting a specific region based on image characteristics in endoscopic images determined as the non-observation target images;
determining images including the specific region among the non-observation target images as correction target images;
determining a directional relationship between a direction of the imaging device and an anatomical orientation of the human body, based on each correction target image; and
performing direction standardization processing to successively rotate the correction target images sequentially obtained during the insertion of the endoscope based on the directional relationship, thereby standardizing orientations of the correction target images based on the anatomical orientation as a reference.

19. The image processing method according to claim 18, further comprising synthesizing a series of the correction target images subjected to the direction standardization processing into a panoramic image.

20. A non-transitory computer-readable storage medium storing computer-readable instructions that are executable by processing circuitry configured to be connected to an endoscope and are configured to, when executed by the processing circuitry, cause the processing circuitry to perform the image processing method according to claim 18.
